(19) United States
(12) Patent Application Publication
(10) Pub. No.: US 2015/0141529 A1
(43) Pub. Date: May 21, 2015

(54) Method and Apparatus for Treating Centralized Pain

(71) Applicant: Cerephex Corporation, Los Altos, CA (US)

(72) Inventor: Jeffrey B. Hargrove, Bancroft, MI (US)

(21) Appl. No.: 14/400,233

(22) PCT Filed: May 8, 2013
(86) PCT No.: PCT/US2013/040045

§ 371 Date: Nov. 10, 2014

Related U.S. Application Data
(60) Provisional application No. 61/644,049, filed on May 8, 2012.

(51) Int. Cl. A61N 1/36; A61N 2/00; A61B 5/0484; G09B 19/00; A61N 1/04

(52) U.S. Cl. CPC A61N 1/36021; G09B 19/00; A61N 1/0472; A61N 1/36071; A61N 1/36139; A61N 1/36192; A61B 5/0484; A61N 2/00; A61N 1/0531
USPC 514/789; 607/46; 434/236; 607/139; 607/59; 607/3; 600/407; 600/9

(57) ABSTRACT

A method for alleviating centralized pain in human subjects includes assessing the brain of a subject suffering from pain, diagnosing abnormal brain function associated with centralized pain, locating at least one area of abnormal brain function associated with the centralized pain, and alleviating the abnormal brain function by applying a cortical stimulation signal to tissues corresponding to the at least one area of abnormal brain function.

### CROSS-REFERENCES TO RELATED APPLICATIONS

This application is a 371 (US national phase) of PCT/US2013/040045, filed May 8, 2013, which claims priority to U.S. Ser. No. 61/644,049, filed May 8, 2012; and a continuation-in-part of U.S. Ser. No. 13/942,246, filed Jul. 15, 2013; and claims the benefit of PCT/US2009/032639, filed Jan. 30, 2009.

### TECHNICAL FIELD

The present invention relates generally to the treatment of pain. More specifically, the present invention relates to methods and apparatuses for treating pain involving abnormal pain processing functions and mechanisms in the brain.

### BACKGROUND

Nociceptive pain is known to arise from stimulation of peripheral nerve endings. The peripheral nociceptive signal is transmitted through the spinal cord to the brain, where it is processed through numerous pain-processing networks. Descending pathways from the brain to the spinal cord subsequently modulate pain signals, thereby increasing or decreasing pain perception.

However, it is also known that enhanced activation of central pain-processing pathways and networks, through mechanisms such as neuroplastic changes in central neuronal activity and network connectivity, can lead to spontaneous pain in the absence of peripheral nociceptive input. When this occurs, pain is said to have “centralized”, which results in lower pain thresholds, secondary hyperalgesia in uninjured areas, and sustained pain potentiation. Brain-related centralized pain is thought to play a prominent role in chronic pain conditions.

Centralized pain is generally thought of as an outcome of central sensitivity (CS), which is also known as central sensitization, central augmentation, and central hypersensitivity among other terms. CS mechanisms in the brain have been implicated in the pathology of allodynia, which is the term used to describe a condition where pain is caused by a stimulus that does not normally provoke pain. CS mechanisms in the brain have also been implicated in hyperalgesia, which is the term used to describe a condition in which pain perceived from a stimulus is greater than what would normally be expected from that stimulus. Put simply, in central sensitivity the brain magnifies painful stimuli and eventually magnifies even associated non-painful stimuli. As pointed out in Latremoliere and Woolfe (1), because CS results from changes in the properties of neurons in the central nervous system, the pain is no longer coupled, as acute nociceptive pain is, to the presence, intensity, or duration of noxious peripheral stimuli arising from both neuropathic and inflammatory sources. Further, in chronic pain conditions the increased excitability caused by CS far outlasts the initiating noxious stimulus, that is, the nociceptive input that causes the pain to occur in the first place.

Before CS was discovered, typically only two models of pain were contemplated. The first is the aforementioned nociceptive pain model, by which specific pain pathways are activated by peripheral pain stimuli, and the amplitude and duration of the pain experienced is determined entirely by the intensity and timing of the peripheral pain inputs. The second model contemplates gate controls in the central nervous system that open and close, thus enabling or preventing pain. Medical science now recognizes CS as a third and unique model that contemplates neuroplastic changes in the functional properties and network connectivity of the central nervous system. For example, the level of resting brain activity within multiple networks (e.g. functional network connectivity and effective network connectivity) is now known to be associated with spontaneous pain in patients having centralized pain (2, 3). CS leads to reductions in pain threshold, increases in the magnitude and duration of responses to noxious input, and permits normally innocuous inputs to generate pain sensations. In addition, CS is also believed to be relevant in somatic symptoms associated with painful conditions, including but not limited to fatigue and sleep disorders.

The brain's role in CS is being increasingly revealed and understood in neuroscience, due in large part to the advent of functional brain imaging technologies. For example, Lee et al. (4) used functional magnetic resonance imaging (fMRI) to examine the extent to which brain activity contributes to the maintenance of CS in humans. When the intensity of pain during CS was matched to the intensity of pain during normal states, activity within the brainstem, including the mesencephalic pontine reticular formation and the anterior thalami, remained at an increased level during CS. Regarding brain areas related to the consequence of increased pain perception during CS, cortical activity, mainly in the primary somatosensory area, has been significantly correlated with the intensity of pain attributable to both the force of noxious stimulation used, and the state in which noxious stimulation was applied.

Borsook et al. (5) reviewed the literature on brain activity using neuroimaging technologies. Their review details evidence of alterations in multiple sub-cortical and cortical processing mechanisms. Those alterations include sensory, emotional/affective, cognitive, and modulatory systems that are present in chronic pain. The authors note these findings provide evidence that increases understanding of the importance of the role of numerous brain regions in the centralization of pain and the contributions of those regions to the altered brain states associated with chronic pain conditions. Similarly, Schweinhardt and Bushnell (6) review neuroimaging evidence of the active and enhanced modulatory role that the brain plays in pain processing in chronic pain patients. Schwienhardt and Bushnell also cite findings that brain activations in chronic pain involve brain circuitry not normally activated by acute nociceptive pain.

Because of this emerging understanding, the role of CS is increasingly being shown to be pathological in seemingly unrelated chronic pain conditions and syndromes including fibromyalgia, complex regional pain syndrome, phantom pain, and migraine headaches. Yunus (7) identifies no less than 14 common syndromes that lack structural pathology yet have CS as a common mechanism. These conditions further include chronic fatigue syndrome, irritable bowel syndrome, tension-type headaches, temporomandibular disorder, myofascial pain syndrome, regional soft-tissue pain syndrome, restless leg syndrome, periodic limb movements in sleep, multiple chemical sensitivity, primary dysmenorrhea, female urethral syndrome, interstitial cystitis, and post-traumatic stress disorder. Yunus also notes that CS may play a significant role in the pain associated with depression and in Gulf War Syndrome.

Giesecke et al. (8) used fMRI to demonstrate augmented central pain processing in patients with idiopathic chronic low back pain and fibromyalgia. Indeed, when equal levels of mechanical pressure intended to elicit a painful response were applied to patients and to normal controls, patients with chronic low back pain and fibromyalgia experienced significantly more pain and showed more extensive, common patterns of neuronal activation in pain-related cortical areas of the brain than did the controls. Thus, CS may play an important role in persons with chronic low back pain that persists without identifiable physical pathology.

The role of CS in persistent inflammatory conditions is also gaining recognition. In Gwilym et al. (9), fMRI illustrated significantly greater brain activation in osteoarthritis (OA) patients in response to stimulation of their referred pain areas (i.e. areas where pain persists but do not exhibit OA or related inflammation) compared with healthy controls, and the magnitude of this activation positively correlated with the extent of neuropathic-like elements to the patient's pain. The role of CS in osteoarthritis has been the subject of several other investigations (10, 11, 12). As detailed in Imamura et al. (13), the refractory, disabling pain associated with knee OA is usually treated with total knee replacement. However, a comparison of OA patients with healthy normal controls showed patients with knee OA had significantly lower pressure pain thresholds (PPT) over widespread evaluated structures beyond the knee. The lower PPT values were correlated with higher pain intensity, higher disability scores, and with poorer quality of life. This suggests that pain in these patients might be more associated with CS than with peripheral inflammation and injury. As the authors point out, the implications of the role of CS, and its potential for modulation, may provide exciting and innovative cost effective therapeutic tools to control pain, reduce disability, and improve quality of life in knee OA patients.

Yet, the treatment of CS is a challenging task. As stated by Latremoliere and Woolfe (1), “The complexity is daunting because the essence of central sensitization is a constantly changing mosaic of alterations in membrane excitability, reductions in inhibitory transmission, and increases in synaptic efficacy, mediated by many converging and diverging molecular players on a background of phenotypic switches and structural alterations.” Some centrally-acting pharmaceutical agents such as gabapentin (14,15), ketamine (16), propofol (17) and anti-tumor necrosis factor alpha (TNF-alpha) therapy (18), just to name a few, have evidence of efficacy in treating CS. The patent literature has examples in the art of pharmaceutical use as a therapeutic agent for treating CS. For example, the use of dimiracetam for treatment of hyperalgesia and allodynia caused by central sensitization in chronic pain has been taught. Further, compounds associated with (R)-2-acetamido-N-benzyl-3-methoxypropionamide have been taught for use in treating central neuropathic pain, including “neurological disorders characterized by persistence of pain and hypersensitivity in a body region.”

Another relevant consideration is that analyses of numerous brain imaging and functional measures, including electroencephalographic (EEG) measures (19), have been shown to produce measures related to brain networks and functional connectivity that correlate to findings produced by fMRI imaging (20). Thus, the presence of brain activity associated with CS, and hence centralized pain, can be determined using EEG measures and analysis.

The following citations are incorporated by reference in their entirety:
1. “Central sensitization: a generator of pain hypersensitivity by central neural plasticity”, Latremoliere A, Woolf C J. J Pain. 2009 September; 10(9):895-926.
2. “Intrinsic brain connectivity in fibromyalgia is associated with chronic pain intensity”, Napadow V, LaCount L, Park K, As-Sanie S, Clauw D J, Harris R E. Arthritis Rheum. 2010 August; 62(8):2545-55.
3. “Disrupted functional connectivity of the pain network in fibromyalgia”, Cifre I, Sitges C, Fraiman D, Muñoz M Á, Balenzuela P, González-Roldán A, Martínez-Jauand M, Birbaumer N, Chialvo D R, Montoya P. Psychosom Med. 2012 January; 74(1):55-62.
4. “Identifying brain activity specifically related to the maintenance and perceptual consequence of central sensitization in humans”, Lee M C, Zambreanu L, Menon D K, Tracey I. J Neurosci. 2008 November 5; 28(45):1142-9.
5. “A key role of the basal ganglia in pain and analgesia—insights gained through human functional imaging”, Borsook D, Upadhyay J, Chudler E H, Becerra L. Mol Pain. 2010 May 13; 6:27.
6. “Pain imaging in health and disease—how far have we come?”, Schweinhardt P, Bushnell M C. J Clin Invest. 2010 November 1; 15(11):3788-97.
7. “Fibromyalgia and overlapping disorders: the unifying concept of central sensitivity syndromes”, Yunus M B. Semin Arthritis Rheum. 2007 June; 36(6):339-56.
8. “Evidence of augmented central pain processing in idiopathic chronic low back pain”, Giesecke T, Gracely R H, Grant M A, Nachemson A, Petzke F, Williams D A, Clauw D J. Arthritis Rheum. 2004 February; 50(2):613-23.
9. “Psychophysical and functional imaging evidence supporting the presence of central sensitization in a cohort of osteoarthritis patients”, Gwilym S E, Keltner J R, Warnaby C E, Carr A J, Chizh B, Chessell I, Tracey I. Arthritis Rheum. 2009 September 15; 61(9):176-34.
10. “Lessons from fibromyalgia: abnormal pain sensitivity in knee osteoarthritis”, Bradley L A, Kersh B C, DeBerry J J, Deutsch G, Alarcon G A, McLain D A. Novartis Found Symp. 2004; 260:258-70.
11. “Sensitization in patients with painful knee osteoarthritis”, Arendt-Nielsen L, Nie H, Laursen M B, Laursen B S, Madeleine P, Simonsen O H, Graven-Nielsen T. Pain. 2010 June; 149(3):573-81.
12. “Pain mechanisms in osteoarthritis: understanding the role of central pain and current approaches to its treatment”, Mease P J, Hanna S, Frakes E P, Altman R D. J Rheumatol. 2011 August; 38(8): 1546-51.
13. “Impact of nervous system hyperalgesia on pain, disability, and quality of life in patients with knee osteoarthritis: a controlled analysis”, Imamura M, Imamura S T, Kaziyama H H, Targino R A, Hsing W T, de Souza L P, Cutait M M, Fregni F, Camanho G L. Arthritis Rheum. 2008 October 15; 59(10):1424-31.
14. “Pharmacological modulation of pain-related brain activity during normal and central sensitization states in humans”, Iannetti G D, Zambreanu L, Wise R G, Buchanan T J, Huggins J P, Smart T S, Vennart W, Tracey I. Proc Natl Acad Sci USA. 2005 December 13; 102(50):18195-200.
15. “Chronic oral gabapentin reduces elements of central sensitization in human experimental hyperalgesia”, Gottrup H, Juhl G, Kristensen A D, Lai R, Chizh B A, Brown J, Bach F W, Jensen T S. Anesthesiology. 2004 December; 101(6):350-8.
16. “Pharmacodynamic profiles of ketamine (R)- and (S)- with 5-day inpatient infusion for the treatment of complex regional pain syndrome”, Goldberg M E, Torjman M C, Schwartzman R J, Mager D E, Wainer I W. Pain Physician. 2010 July; 13(4):379-87.
17. “Analgesic and antihyperalgesic properties of propofol in a human pain model”, Bandschapp O, Filitz J, Ihmsen H, Berset A, Urwyler A, Koppert W, Ruppen W. Anesthesiology. 2010 August; 8(2):421-8.
18. “TNF-alpha and neuropathic pain—a review”, Leung L, Cahill C M. J Neuroinflammation. 2010 April 16; 7:27.
19. “Functional connectivity: the principal-component analysis of large (PET) data sets”, Friston K J, Frith C D, Liddle P F, Frackowiak R S. J Cereb Blood Flow Metab 1993; 13:5-14.
20. “Electrophysiological signatures of resting state networks in the human brain”, Mantini D, Perrucci M G, Del Gratta C, Romani G L, Corbetta M. Proc Natl Acad Sci USA. 2007 August 7; 104(32):2670-5.

### SUMMARY

A method is provided for alleviating centralized pain. The method may include the steps of assessing the brain of a subject suffering from pain, diagnosing one or more brain conditions associated with centralized pain, and locating at least one area of abnormal brain measure associated with an abnormal brain condition producing the centralized pain. Also, a cortical stimulation signal may be applied to at least one area of abnormal brain measure to alter the one or more brain conditions in such a way as to alleviate the centralized pain.

A method is provided for assessing a brain to determine the presence of centralized pain, which may include the use of a brain response test comprising one or more neuroimaging tests performed before, during and after the application of any one or more sensory stimulations to cause one or more brain responses.

A method is provided for predicting symptom severity in individuals having centralized pain, which may include the steps of executing brain response testing on a subject, obtaining brain measures associated with centralized pain in the subject by analyzing findings from the brain response testing, and correlating the brain measures to measures of symptom severity.

A method is provided for determining the effect of therapeutic intervention in alleviating symptoms of centralized pain, which may include the steps of executing brain response testing on a subject, obtaining brain measures associated with centralized pain in the subject by analyzing findings from the brain response testing, and correlating the brain measures to measures of the effect of therapeutic intervention.

A method is provided for determining points for application of cortical stimulation for alleviating centralized pain by analyzing a brain response test.

A method is provided for optimizing an intervention for alleviating centralized pain, which may include the steps of collecting brain measures associated with one or more abnormal brain conditions associated with centralized pain during a time period at or near the time of a therapeutic intervention, analyzing the brain measures by computational algorithms to determine measures and statistics associated with the brain measures, and using the measures and statistics to modify parameters of an intervention for the purposes of optimizing therapeutic benefit.

An apparatus for diagnosing and treating centralized pain is provided, which may comprise a cap configured to be worn on a subject's head and a series of interconnectable flexible bands configured to include arms and tabs that connect when placed on a head to form a means to fit the shape of the head.

An apparatus for diagnosing and treating centralized pain is provided, which may comprise a cap configured to be worn on a subject's head and interconnected flexible bands that form the cap and are configured to position one or more electrodes to measure one or more EEG signals from a subject and/or deliver one or more cortical stimulation signals to a subject.

A method is provided for delivering one or more cortical stimulation signals to a subject that may include the use of an apparatus comprising one or more electrodes including one or more ground electrodes positioned in flexible bands configured to include arms and tabs that connect when placed on a head to form a cap to fit the shape of the head.

An abnormal brain function diagnostic and treatment apparatus is provided, which may comprise a cap configured to be worn on a subject's head and may also comprise one or more electrodes positioned to contact a subject's head when the subject is wearing the cap, and the cap may also comprise a memory configured to store information.

A method is provided for preventing a cap from being used more than a predetermined number of times. The method may include providing a cap identifier on the cap and an allowed use indicator representing a predetermined number of allowed uses for the cap. The method also may include gaining access to stored information in the cap identifier and the cap allowed use indicator, comparing the stored information against a database of information on caps that have previously been used, and comparing the number of allowed uses indicated in the cap allowed use indicator against the previous cap use number stored in the cap information database. The method also may include configuring an apparatus for diagnosing and treating centralized pain to allow use of the cap only if the cap information database indicates that the previous cap use number is less than the number of allowed uses indicated by the cap allowed use indicator.

A method is provided for altering brain network connectivity in a subject. The method may include the steps of identifying a target network in the subject's brain, identifying a target region of tissues, and stimulating the target region of tissues to alter the brain network connectivity.

Additional advantages and novel features of the invention will be set forth in part in the description that follows, and in part will become more apparent to those skilled in the art upon examination of the following or upon learning by practice of the invention.

### BRIEF DESCRIPTION OF THE DRAWINGS

These and other features and advantages will become apparent to those skilled in the art in connection with the following detailed description, drawings, photographs, and appendices, in which:

FIG. 1 is a flow chart depicting a treatment method;

FIG. 2 is a schematic diagram showing an apparatus for treating a brain-related chronic pain disorder;

FIG. 3 is a schematic diagram showing a signal generating circuit of the apparatus of FIG. 2;

FIG. 4 is a schematic diagram showing an embodiment of an apparatus for treating a brain-related chronic pain disorder and showing a therapy cap of the apparatus and a subject's head cut-away to reveal electrical stimulation signal paths relative to target areas of the subject's brain tissue;

FIG. 5 is a schematic diagram showing an embodiment of an apparatus for treating a brain-related chronic pain disorder and showing a therapy cap of the apparatus cut-away to reveal an RFID chip carried by the cap;

FIG. 6 is a schematic diagram showing an embodiment of an apparatus for treating a brain-related chronic pain disorder and showing a therapy cap of the apparatus cut-away to reveal a programmable memory circuit carried by the cap;

FIG. 7 is a schematic diagram showing an embodiment of an apparatus for diagnosing and treating centralized pain;

FIG. 8 is a schematic diagram showing a signal generator circuit embodiment of the apparatus of FIG. 7;

FIG. 9 is a schematic diagram showing an embodiment of a cap apparatus configured to be worn on a subject's head;

FIG. 10 is another schematic diagram showing an embodiment of a cap apparatus configured to be worn on a subject's head;

FIG. 11 is a flow diagram of a method of alleviating centralized pain by diagnosing brain conditions associated with centralized pain and applying a cortical stimulation signal;

FIG. 12 is a flow diagram of a method of diagnosing centralized pain based on analyzing findings from one or more brain response tests;

FIG. 13 is a flow diagram of a method of determining the presence of centralized pain by determining brain response test abnormalities consistent with centralized pain;

FIG. 14 is a flow diagram of a method of predicting symptom severity by correlating measures of a brain response test to measures of symptom severity;

FIG. 15 is a flow diagram of a method of determining the effect of therapeutic intervention by correlating measures of a brain response test to measures of the effect of therapeutic intervention;

FIG. 16 is a flow diagram of a method of determining points for application of cortical stimulation using analysis of findings from a brain response test;

FIG. 17 is a flow diagram of a method of assessing the effectiveness of a therapeutic intervention;

FIG. 18 is a flow diagram of a method of transferring cortical stimulation signal parameters via electronic means;

FIG. 19 is a flow diagram of a method of optimizing therapeutic benefit of a cortical stimulation signal;

FIG. 20 is a flow diagram of a method of forming and positioning a cap to deliver cortical stimulation signals;

FIG. 21 is a flow diagram of a method of preventing a cap from being used more than a predetermined number of times;

FIG. 22 is a flow diagram of a method of alleviating centralized pain by applying a cortical stimulation signal in combination with treatment of another coexisting physical condition;

FIG. 23 is a flow diagram of a method of a method of alleviating centralized pain by applying a cortical stimulation signal in combination with administering a pharmaceutical agent;

FIG. 24 is a flow diagram of a method for altering network connectivity in a brain by applying a cortical stimulation signal; and

FIG. 25 is a flow diagram of a method for altering network connectivity in a brain by administering a pharmaceutical agent and applying a cortical stimulation signal.

### DETAILED DESCRIPTION

In the following description of the disclosed apparatus and methods, the term “centralized pain” is intended to mean any form of pain, whether chronic or acute, that is enhanced in its characteristics; such as magnitude, duration and scope; due to abnormal brain activity associated with pain processing. Such brain activity may include, but is not limited to, central sensitivity and network connectivity.

The term “central sensitivity” is intended to mean any central nervous system condition pathologically related to hyperalgesia, allodynia, reductions in pain threshold, increases in the magnitude and duration of responses to noxious input, results in normally innocuous inputs to generate pain sensations, or results in non-painful symptoms associated with increases in central nervous system responsiveness. Central sensitivity is also known by alternate terms that include but are not limited to “central sensitization”, “central pain”, “central augmentation,” and “central hypersensitivity”.

Central sensitivity is not a manifestation or cause of an individual symptom or condition. Instead, central sensitivity results in a worsening of the effect or magnitude of one or more symptoms because of a central nervous system condition that is independent of the cause of the one or more symptoms per se. Thus, any method of treatment of central sensitivity is fundamentally different from treatment of a specific symptom. For example, treatment of pain augmentation by central sensitivity is inherently different than treatment of pain under traditional nociceptive models of pain.

The terms “network connections” and “network connectivity” are intended to mean various forms of relationships between brain regions involved in processing of information such as pain. For example, “functional connectivity” refers to a statistical correlation between the activities of different brain regions. “Effective connectivity” denotes not simply a statistical but a causal influence between two brain regions.

The term “alleviate” or “alleviating” is intended to mean the act of reducing, making less severe, mitigating, treating, or eliminating a condition and/or its symptoms for any period of time.

Except where the context requires otherwise, the term “comprise” and variations of the term, such as “comprising”, “comprises” and “comprised” are not intended to be exclusive. Where, for example, a form of the word “comprise” is used to refer to one or more additives, components, integers or steps; its use is not intended to exclude other additives, components, integers or steps.

Where the terms “integral” or “integrated” are used to describe a relationship between two or more elements, the terms are intended to indicate that such elements are joined together in a manner that does not allow separation of elements from one another without diminishing or destroying a function of one or more of the elements.

The term “stimulating” is intended to mean the transmitting of any energy signal generated by a stimulation device such as an electrical stimulator, or by a magnetic stimulator such as a transcranial magnetic stimulator, to the brain of a subject for the purpose of influencing any function or physiological state of the subject's brain that is at least one part of a pathway of centralized pain.

The term “stimulation signal” is intended to mean any energy signal used in the process of stimulating a tissue such as a brain. Other terms used to refer to such a signal may include but are not limited to “cortical stimulation”, “neuromodulation” and “neurostimulation”.

The term “neuroimaging test” is intended to mean any medical test that provides visual indication, measures, or other data that can be used to make an assessment about central nervous system function, including brain function. Types of tests that the term “neuroimaging test” may be used to refer to include, but are not limited to, magnetic resonance imaging, computer aided tomography, positron emission tomography, or single photon emission computed tomography, and may also include brain electrical function tests such as electroencephalography or magnetoencephalography.

The term “brain activities” is intended to refer to any brain activities that are known in the art to be associated with central sensitivity. Such brain activities are intended to include, but are not limited to abnormal function, abnormal response, abnormal regions of activation, abnormal network connectivity, abnormal release of neurochemicals, abnormal uptake of neurochemicals, abnormal electrical activity, or abnormal metabolism.

The term “brain function” is intended to mean any action or process of a brain in the brain's normal course of operation.

The term “spectral segments” is intended to mean frequency components of an electrical signal that includes individual frequency components, and in the case of an EEG signal, that includes groupings of frequency components commonly known as “frequency bands”, such bands including, but not limited to the “delta” band (nominally 1-3.5 hertz), the “theta” band (nominally 4-7.5 hertz), the “alpha” band (nominally 8-12 hertz) and the “beta” band (nominally 12.5-25 hertz).

The term “resting EEG” is intended to mean electroencephalogram signals that are collected with the subject's eyes either open or closed and during periods of no significant physical activity, mental activity, or any other form of engagement that may cause the brain to be stimulated significantly or engaged in elevated brain function.

The term “biopotential” is intended to mean any electrical signal representing a measurable physiological function or state. Such electrical signals may include, but are not limited to, electroencephalogram signals, magnetoencephalogram signals, electromyogram signals, signals representing measures of vital signs and signals representing measures of organ function.

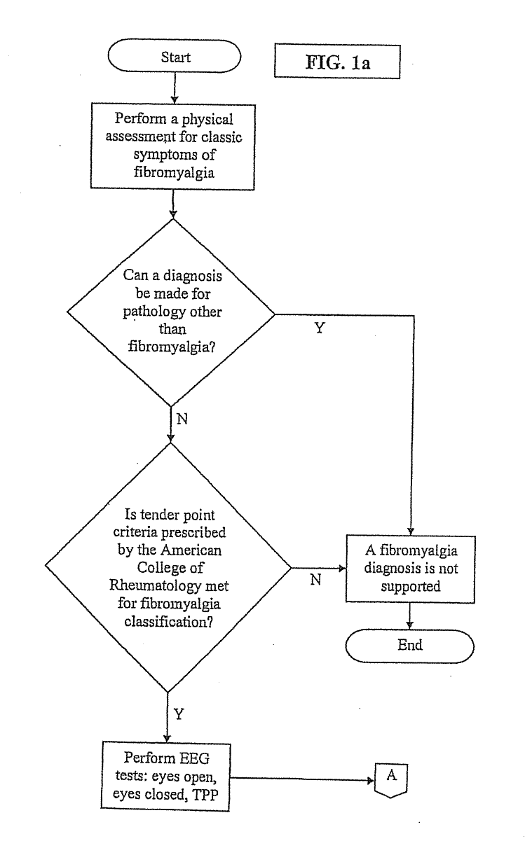

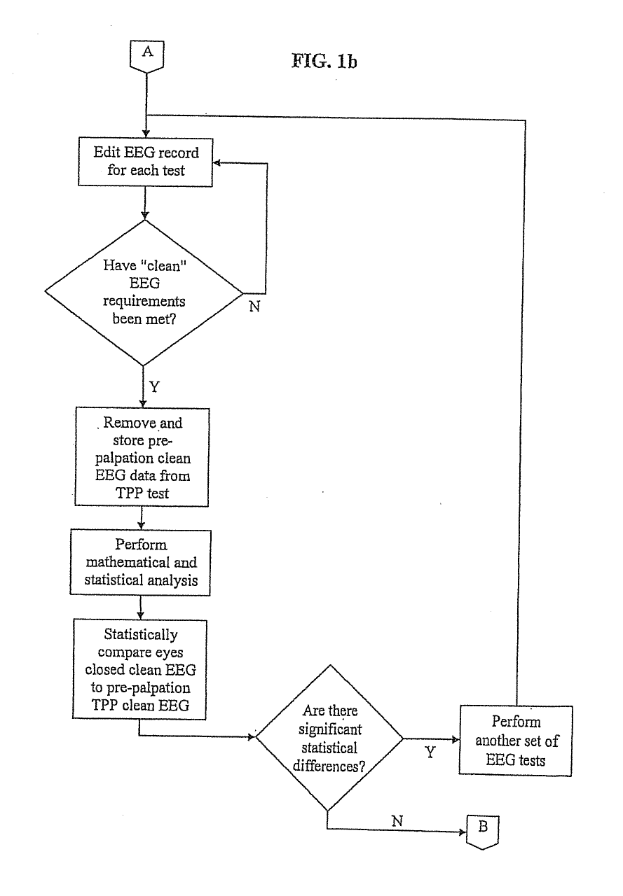

With reference to FIGS. 1a-1e, a method is provided for treating a brain-related chronic pain disorder. The method includes assessing the brain function of a subject suffering from chronic pain, diagnosing a chronic pain-related abnormal brain condition, locating at least one area of abnormal brain activity associated with the abnormal brain condition and mitigating the abnormal brain activity by applying a neuromodulation signal to tissues corresponding to the at least one area of abnormal brain activity. Alternatively, the neuromodulation signal comprises waveforms designed to minimize tissue impedance while effecting noninvasive neuromodulation. Treatment effect is realized when abnormal brain function has been improved or corrected.

A physical assessment may first be performed of a human subject presenting with a complaint of symptoms characteristic of a chronic pain condition such as fibromyalgia. The physical assessment may include, among other things, a determination of chronic widespread pain, sleep difficulty, fatigue, morning stiffness of the muscles and joints, cognitive difficulty, and other symptoms associated with the condition. The physical assessment may also include tests performed to exclude various non-fibromyalgia conditions as the cause of the symptoms. Such further testing may include palpation of tissues to elicit a painful response, such as palpation of fibromyalgia tender points in the manner prescribed by the American College of Rheumatology, with such palpation being performed to determine whether the subject has an abnormal sensitivity to pain.

In the absence of an alternate, non-fibromyalgia diagnosis, an electroencephalogram (EEG) test may be performed in addition to the physical assessment, whereby the EEG test is performed utilizing methods and apparatus well known in the art. Specifically, the subject may be made comfortable by, for example, being seated, or reclined. Preparation of the scalp in accordance with commonly followed procedures for performing a clinical EEG may be done by a person of sufficient competence. EEG electrodes may then be adapted to be worn on the scalp, preferably in scalp locations identified as the “International 10-20” standard sites, using common methods of affixing the electrodes such that they rest on or otherwise contact tissues.

While any number of electrodes may be used, a preferred number is either 19 or 24, in accordance with the number of electrode sites used to construct various independent databases utilized to represent the EEG of a healthy normal population, and to facilitate quantitative assessment (qEEG) of the subject's EEG. Methods involving qEEG include a number of mathematical analyses utilized to make statistical comparisons between the subject's qEEG and a database of qEEGs of either healthy normal individuals' brain functions or the brain functions of individuals suffering from chronic pain related brain function conditions.

Records of the subject's EEG from each electrode site may then be acquired under the conditions of both their eyes being closed and their eyes being open, with each condition producing a separate data record. In other words, an “eyes open” EEG record may be obtained, which includes EEG data obtained from each electrode site while the subject's eyes are open and an “eyes closed” EEG record may be obtained, which includes EEG data obtained from each electrode site while the subject's eyes are closed. Preferably, a minimum of five minutes of EEG data may be obtained from each electrode site for each “eyes open” EEG record and a minimum of five minutes of EEG data may be obtained from each electrode site for each “eyes closed” EEG record to assure that enough EEG data is recorded to produce statistically significant samples from each electrode site, both with the subject's eyes open and with the subject's eyes closed. This is further described below.

Preferably, an additional test may be performed in which at least one additional EEG record is made that includes EEG data obtained at each electrode site while the subject's eyes are closed. In this test, henceforth referred to as a “tender point palpation (TPP) test”, a number of tender points on the subject's body, preferably ranging between one and 18, are identified and serially palpated with an algometer. Preferably, four tender points may be chosen, and, preferably, those four points include tender points adjacent the right and left lateral epicondyle of the arms approximately two centimeters distal of the elbows, and tender points adjacent the right and left costochondral junctions of the second rib.

The TPP test may be executed by acquiring an EEG record (“TPP” EEG record) including EEG data obtained from the electrode sites for a first tender point by first commencing the acquisition of EEG data and then, a short period of time later, commencing palpation of the first tender point. Preferably, the period of time between the commencement of data acquisition and the commencement of palpation of the first tender point may be between one and 300 seconds. Palpation of the first tender point may be accomplished by pressing on the tender point—preferably pressing or palpating through the use of an algometer, and preferably at a rate of approximately one kilogram per centimeter squared per second, until the subject reports a painful sensation or until reaching a pressure of 4 kilograms per centimeter squared—whichever occurs first. Preferably, palpation pressure may be removed as soon as the subject reports a painful sensation. A record is made of the amount of the pressure being applied at the moment the subject reports a painful sensation.

Further according to the TPP test method, the recording of the “eyes closed” EEG may continue for a period of time after release of palpation pressure, preferably between 1 and 300 seconds, and most preferably, for at least 60 seconds. Following this period, a second and subsequent tender point may be serially palpated with an algometer in the same manner as described for the first, with “TPP” EEG records being recorded for each by recording the “eyes closed” EEG for each site in the manner described with regard to obtaining the “TPP” EEG record for the first site. This process may be repeated for each chosen tender point. Accordingly, the resulting EEG data record includes the “TPP” EEG records acquired for each chosen tender point.

The “TPP” EEG records may be acquired for a period of time that is sufficient to extract from each “TPP” EEG record a minimum of 60 seconds of “clean” EEG data, that is, data free of extraneous electrical noise such as that from electromyographic movement. Preferably, all EEG records (“eyes open” EEG records, “eyes closed” EEG records, and “TPP” EEG records) may be individually edited to provide from each EEG record a minimum of 60 seconds of clean EEG. Preferably, the clean data is obtained to present a high degree of statistical consistency. Such measures as “Split-Half” reliability, which is the ratio of variance between the even and odd seconds of the time series of selected clean EEG; and “Test Re-test” reliability, which is the ratio of variance between the first half and the second half of the selected clean EEG segments may be used. Preferably, clean EEG data is obtained such that measures of these ratios are a minimum of 0.95 and 0.90 respectively, which is consistent with levels of reliability commonly published in EEG literature.

With regard to the TPP test method, clean data includes only that EEG data acquired after palpation of a tender point, and does not include any EEG data acquired during the palpation of a tender point. In addition, to assess the stability of a “TPP” EEG record, EEG data acquired before palpation of a tender point may be removed, edited and statistically compared to like data in the “eyes closed” EEG record obtained from the “eyes closed” EEG test. Stability of the “eyes closed” and “TPP” EEG records is indicated by a finding that there is no statistically significant difference between the “eyes closed” EEG record and the pre-palpation portion of the “TPP” EEG record. A contrary finding indicates instability and a need to repeat the EEG tests.

Further to the method, and in the preferred embodiment, clean “eyes open”, “eyes closed”, and “PPT” EEG records may be then mathematically analyzed for various time domain and frequency domain parameters of their respective electrical signals. These analyses may include, but are not limited to voltage analysis, current analysis, voltage and current analysis, frequency spectrum analysis using Fast Fourier transform analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, and localization of electrical activity in the brain using inverse EEG computation analysis.

Findings from the aforementioned analyses may then be statistically compared to the same parameters determined from “eyes open”, “eyes closed”, and “PPT” EEG records taken from an age and gender matched database of healthy normal individuals. Such statistical analyses may include, but are not limited to deviations from a standard normal distribution. Findings of statistically significant abnormal deviation, or lack thereof, may then be presented in a graphical or numerical format for analysis by a competent health care professional or person of similar expertise.

EEG abnormalities consistent with those observed in a sample population of fibromyalgia patients may include, but are not limited to one or more of the following: (1) an overall reduction in EEG power across all spectra in either of the “eyes open” or “eyes closed” conditions; (2) statistically significant low EEG power levels in frontal or temporal regions of any of the delta (1-3.5 hertz), theta (4-7.5 hertz) or alpha (8-12 hertz) frequency segments of EEG for the “eyes closed” condition; (3) statistically significant low coherence among the frontal EEG sites for the delta or theta EEG segments in either of the “eyes closed” or “eyes open” conditions; (4) statistically significant high relative beta (12.5-25 hertz) absolute power in the parietal region of the brain for either of the “eyes closed” or “eyes open” conditions. The magnitude of statistical variation considered statistically “significant” may vary depending on the application. For example, in research, a difference between a sample and a population measure generally has to have a p-value of 0.01 or less for the difference to be considered statistically “significant”. However, in clinical application statistically significant differences may be declared with p-values at the 0.1 level or less.

Further EEG abnormalities consistent with those observed in a sample population of fibromyalgia patients, and drawn particularly to the TPP test method, may include but are not limited to a finding of (1) a statistically significant increase in EEG absolute power, particularly in the alpha and beta segments, in the parietal, occipital, and temporal areas of the brain as compared to the “eyes closed” EEG record (“eyes closed” EEG findings without tender point palpation) for the same subject; or (2) a statistically significant increase in coherence in the alpha or beta segment of EEG. The following are the results of tests of the predictive value of TPP sensitivity analysis, obtained when TPP testing was utilized on 19 fibromyalgia patients and compared to TPP testing done on nine healthy normal controls:

| | | | Positive |
|---|---|---|---|
| Test Criterion for Making a Diagnosis | Sensi- | Speci- | Predictive |
| of Fibromyalgia | tivity | ficity | Value |
| An increase* in alpha EEG of at least 20% | 63% | 89% | 92% |
| in at least one occipital or parietal site | | | |
| An increase in alpha EEG of at least | 84% | 78% | 89% |
| 20% in at least one temporal site | | | |
| The total regions of increase in alpha EEG | 74% | 100% | 100% |
| of at least 20% are greater than two | | | |
| Alpha EEG coherence increases by at | 84% | 88% | 94% |
| least 20% in at least 30 out of 171 | | | |
| possible site combinations | | | |
| At least two (2) positive findings occur in | 90% | 100% | 100% |
| any of the four previous tests | | | |

*Based on comparison of TPP EEG data against eyes closed EEG data

A diagnosis of fibromyalgia may be made when physical assessment findings that support a diagnosis of fibromyalgia are augmented by making a quantitative assessment including but not necessarily limited to a statistical comparison between the subject's qEEG and a database of quantitative assessments of either healthy normal individuals or individuals suffering from a chronic pain related abnormal brain function condition such as fibromyalgia. In the preferred embodiment, statistical findings that support a diagnosis of fibromyalgia may include, but are not necessarily limited to, (1) an abnormal finding resulting from the TPP test, preferably a finding of a statistically significant increase in EEG absolute power, and particularly in the alpha and beta segments, in the parietal, occipital, and temporal areas of the brain as compared to the “eyes closed” findings without tender point palpation for the same subject; and preferably (2) an abnormal finding resulting from the “eyes closed” EEG test, preferably statistically significant low EEG power levels in frontal or temporal regions of any of the delta, theta or alpha frequency segments of EEG for the “eyes closed” condition, and most preferably with an additional finding of statistically significant low coherence among the frontal EEG sites for the delta or theta EEG segments. Alternately, fibromyalgia may be diagnosed by statistically comparing a subject's one or more qEEG parameters to like qEEG parameters obtained from at least one healthy normal individual; then comparing the one or more deviations to deviations detected in a sample population of known fibromyalgia patients.

Clean EEG records from a subject may be mathematically analyzed for various time domain and frequency domain parameters of their electrical signals, consistent with analysis techniques already described, and then findings from these mathematical analyses may be statistically compared to like parameters taken from an age and gender matched database of healthy normal individuals or individuals known to have fibromyalgia. The statistical comparisons may include, but are not limited to deviations from a standard normal distribution of like EEG measures associated with members of a database of healthy normal individuals or individuals known to have fibromyalgia. The results of those comparisons may then be presented in a graphical or numerical format for analysis by a competent health care professional or person of similar expertise for the existence of statistically significant abnormal deviations, or the lack thereof. A finding in support of a fibromyalgia diagnosis would be supported if there is an absence of any significant deviation between measures from a subject's clean EEG and those from a database comprising individuals known to have fibromyalgia.

Analyses of clean EEG from a subject may be statistically correlated to measures of symptom severity. As previously described, analysis findings may be mathematically analyzed for various time domain and frequency domain parameters of electrical signals. A number of measures of the magnitude of deviation from standard normal distributions of either healthy normal EEG or known fibromyalgia patient EEG can be determined. The magnitudes may be presumed to be related to the severity of the condition and may be statistically correlated to such symptom measures that may include, but are not limited to, tender point pain pressure thresholds as determined by an algometer, and various other indices of pain derived from the algometry measures (e.g. the sum of all 18 tender point pain tolerance measures, the average of all 18 tender point pain tolerance measures, etc.). Such analysis has utility in both predicting symptom severity in individuals with fibromyalgia, and in determining the effect of therapeutic intervention to correct or manage symptoms of fibromyalgia.

EEG analyses may also be used for determining the location of abnormal brain activity and further for determining points for application of neuromodulation.

Treatment may include the application of a noninvasive neuromodulation signal in a manner designed to correct abnormal brain function identified in accordance with the aforementioned EEG analyses. Suitable noninvasive neuromodulation techniques are disclosed in applicant's U.S. patent application Ser. No. 11/490,255 (issued as U.S. Pat. No. 7,715,910) and applicant's International Patent Application Ser. No. PCT/US2008/72395, which are incorporated herein by reference. The noninvasive neuromodulation signal may comprise those waveforms designed to minimize tissue impedance, such as an “amplitude modulated pulse width modulated” (AMPWM) signal. An AMPWM signal utilizes a high frequency carrier signal that is amplitude modulated by a low frequency neuromodulation signal. The carrier signal is of sufficiently high frequency so as to be less attenuated by the impedance of tissues due to their capacitive reactance. The frequencies used in the neuromodulation signal are lower than the frequency of the carrier signal, and are chosen to provide therapeutic benefit. By using the neuromodulation signal to amplitude modulate the carrier signal, and subsequently applying the combined signal to tissues, the neuromodulation signal is less attenuated by the impedance of the tissue permitting greater penetration of electrical current and field. The carrier signal is further pulse width modulated in an AMPWM signal to control the time averaged current, and hence the power of the signal delivered to the tissues. An apparatus for generating and delivering an AMPWM signal includes any number of electric signal generating devices capable of generating and altering the parameter aspects of an AMPWM signal. Various forms of an AMPWM signal and apparatus for generating an AMPWM signal are disclosed in the applicant's U.S. application Ser. No. 11/490,255 (issued as U.S. Pat. No. 7,715,910) and applicant's International Patent Application Ser. No. PCT/US08/72395. Reports on the results of a double-blind, placebo-controlled study of the efficacy of this treatment are summarized below:

Thirty-nine (39) active treatment (AT) fibromyalgia patients and 38 comparable placebo control patients completed non-invasive neuromodulation treatment, applied twice a week for 11 weeks. The placebo condition (PL) was created by not delivering the non-invasive neuromodulation signal. Both number of tender point (defined by the American College of Rheumatology) and total pain score were evaluated at baseline and end of treatment. Subjects also completed health impact questionnaires (Fibromyalgia Impact Questionnaire [FIQ], Symptom CheckList-90 [SCL-90], Beck Depression Inventory [BDI], and sleep quality) at baseline and end of treatment period, and FIQs in long-term follow up. Primary outcome measures were changes in the number of tender points (TePs) and level of TeP pain, secondary measures were changes in the questionnaire responses.

Analysis of results showed AT patients improved in number of positive TePs, mean 17.4 pre-treatment to 9.9 post-treatment (P<0.001). The between group change was significantly improved (PL −0.2 versus AT −7.4, P<0.001). Sixty-two percent (62%) of the AT group no longer met the tender point criteria for FM classification following treatment. Similarly, the tender point score (TPS) for the AT group improved from 36.7 to 56.4 (P<0.001) whereas the control group got slightly worse, 38.9 to 35.8. The between group change was also significantly improved (PL −3.2 versus AT+19.6, P<0.001). The total FIQ score in the AT group improved from 65.1 to 46.0 (P<0.001). In other measures, the AT group reported 61.8% improvement in sleep quality (P<0.001). Long term follow-up FIQ analysis was done at an average of 16.8 months since discontinuation of treatment (range 12-28 months). There was a continuing long term improvement over baseline values (P<0.001). There were no significant side effects.

Mitigation of abnormal activity is accomplished by generating and applying to the subject an electrical stimulation signal having at least one parameter configured to modulate at least one abnormal aspect of the subject's EEG, which corresponds to at least one statistically significant difference found in the statistical comparison of qEEGs. Such abnormal aspects of the subject's EEG may include, but are not limited to, abnormally high or low amplitudes, abnormal amplitudes in specific frequencies or frequency segments, abnormal spectral power, abnormal relative power, amplitude asymmetry, and abnormally high or low coherence. The application of said electrical stimulation signal, preferably an AMPWM noninvasive neuromodulation signal, may comprise a first step of choosing neuromodulation signal parameters intended to correct abnormal brain function identified in accordance with the aforementioned EEG analyses. These parameters may include, but are not limited to, a choice of the carrier signal frequency, neuromodulation signal frequency, amplitude, waveform, duty cycle, application times, and phase. The step of choosing neuromodulation signal parameters may include identifying a particular signal parameter, such as a frequency from a patient's EEG, that is statistically different than normal, e.g., an EEG frequency that is lower than normal at a particular location. The chosen neuromodulation signal parameters may thus, for example, include a frequency generally equal to that of the abnormally low measured EEG frequency. The step of choosing neuromodulation signal parameters may alternatively or additionally include identifying an area of the brain where the spectral amplitude of an EEG frequency measure is found to be statistically different than normal. This step may further include identifying the direction and magnitude of said spectral amplitude deviation from normal for said statistically different than normal EEG frequency measure. The identifying step may yet further include choosing signal parameters that include frequencies ranging between the frequency of the statistically different than normal measure (F1) and a frequency that is (a) within an approximate range of from 20 Hertz greater than F1 to F1 for the case in which the direction of deviation for F1 is less than normal; or (b) within an approximate range of from 20 Hertz less than F1 to F1 for the case in which the direction of deviation for F1 is greater than normal. The identifying step may further include choosing signal amplitudes and application times that are proportional to the magnitude of deviation from normal for said statistically different than normal EEG frequency measure. The identifying step may further include choosing signal duty cycle so as to provide a signal that cannot be felt by a person when applied. The identifying step may further include choosing a signal waveform that encompasses at least one of the frequencies in the range of F1 plus or minus 20 Hertz. The identifying step may also include applying at least two neuromodulation signals to different areas of the brain, and applying a phase shift between the at least two signals where the phase shift may range between zero and 180 degrees.

The application of a noninvasive neuromodulation signal may further comprise the step of choosing neuromodulation signal application location to provide for application of neuromodulation to tissues corresponding to one or more of the spatial location(s) of abnormal brain function identified by the aforementioned analyses. Signal application may further include the use of electrodes to create a signal path between an electrical stimulation signal source such as an apparatus for generating an AMPWM signal and a stimulating electrode positioned proximate to brain tissues in at least one area of abnormal brain activity, either invasively or non-invasively.

Delivery of the neuromodulation signal may be accomplished by utilizing an electrode set comprising invasive stimulating electrodes positioned on or in near proximity to brain tissues exhibiting abnormal function, i.e., within approximately 20 mm. The electrode set may further comprise an invasive ground electrode positioned such that a vector path between stimulating electrodes and a ground electrode passes through tissues to be stimulated.

Delivery of the neuromodulation signal may be accomplished by utilizing an electrode set comprising one or more non-invasive stimulating electrodes adapted to be worn by a subject such that the stimulating electrodes rest on the scalp in proximity to brain tissues exhibiting abnormal function. The electrode set may further comprise a non-invasive ground electrode adapted to be worn by a subject such that the non-invasive ground electrode rests on the scalp in proximity to brain tissues exhibiting abnormal function; positioned such that a vector path between non-invasive stimulating electrodes and a non-invasive ground electrode passes through or in near proximity to tissues to be stimulated.

Delivery of the neuromodulation signal may be accomplished by utilizing an electrode set comprising one or more non-invasive stimulating electrodes adapted to be worn by a subject such that the stimulating electrodes rest on the skin posterior to the cervical vertebrae and in proximity to the vagus nerve. The electrode set may further comprise a non-invasive ground electrode adapted to be worn by a subject such that the non-invasive ground electrode rests on the scalp in proximity to brain tissues exhibiting abnormal function; positioned such that a vector path between non-invasive stimulating electrodes and a non-invasive ground electrode passes through or in near proximity to tissues to be stimulated.

The period of time over which therapeutic intervention takes place may comprise repeated application of a neuromodulation signal for finite duration, with rest time taking place between applications, and total number of applications comprising a finite number. The finite duration may be between one second and 60 minutes; the rest time may be between one minute and seven days; and the total number of applications may be between one application and 300 applications. The number of applications may be proportional to either the extent of abnormal function and/or the time that the abnormal function has been present.

The method of generating described neuromodulation signals may include the use of an apparatus such as that disclosed in the applicant's U.S. patent application Ser. No. 11/490,255 (issued as U.S. Pat. No. 7,715,910), which is assigned to the assignee of the present application and incorporated herein by reference.

The method may include the steps of repeating quantitative assessments such as EEG testing, TPP testing and statistical analysis on a subject, as described herein, following a period of therapeutic intervention on said subject. The method may further comprise statistical comparison of parameters of the repeated statistical analysis to like parameters of the statistical analysis of the subject done before the previous therapeutic intervention was started. Such comparison might include, but is not limited to, paired t-testing statistics, correlation analysis of changes in symptom severity, and subsequent comparison to a database of age and gender matched healthy normal individuals or individuals suffering from a brain related chronic pain condition such as fibromyalgia. These comparisons may be used to assess the effectiveness of the therapeutic intervention, in particular noninvasive neuromodulation, or to determine if an alternate intervention is indicated in the absence of treatment effect from a current therapeutic intervention. The comparisons could also be used to determine if further therapeutic intervention is indicated in the absence of any abnormal findings. The comparisons may further be used to modify neuromodulation signal parameters in accordance with the findings of the repeated quantitative assessment step.

With specific reference to the TPP test, repeat testing may include the application of tender point pressure using, e.g., an algometer, only to the levels required to cause a painful response recorded in the same testing performed before therapeutic intervention.

Further according to the method, EEG data may be acquired at a first location (e.g. a clinical location) and the acquired EEG data transferred via electronic means to another location (e.g. a central analysis location) for the herein described analysis and statistical comparisons to be accomplished. The electronic means of data transfer may include, but isn't limited to means of data transfer across a local area network and/or the Internet. Consequently, analysis and statistical findings may then be transferred from a central analysis location to a clinical location, where they may be used in various ways by a physician or similarly qualified health care professional for the determination of parameters of a neuromodulation signal used for therapeutic intervention and treatment of fibromyalgia.

Further according to the method, EEG data may be acquired at a first location (e.g. a clinical location) and the acquired EEG data transferred via electronic means to another location (e.g. a central analysis location) for a purpose such as increasing the size of various databases of individuals known to be suffering from fibromyalgia, individuals known to be suffering from a chronic pain condition that is not fibromyalgia, and healthy normal individuals.

Further according to the method, neuromodulation signal parameters may be determined at a central analysis location and subsequently transferred as data via electronic means to an apparatus at another location (e.g. a clinical location) provided for delivery of a neuromodulation signal used for therapeutic intervention and treatment of fibromyalgia. The electronic means of data transfer may include, but isn't limited to, means of data transfer across a local area network and/or the Internet.

The steps of application of the neuromodulation signal and repeat measurements and analyses of a subject's EEG may be continued until abnormal brain function, as determined by, for example, EEG analysis, is modulated and/or mitigation or resolution of symptoms of the chronic pain condition (such as fibromyalgia) are achieved.

Alternatively, non-EEG methods of assessing brain function may be utilized to quantify and locate abnormal brain function. Such methods include, but are not limited to positron-emission tomography (PET) scans, magnetic resonance imaging (MRI) testing and single photon emission computed tomography (SPECT) scans.

Alternatively, EEG data may be collected during a therapeutic intervention that includes application of an electrical stimulation signal such as a neuromodulation signal, and that EEG data may be analyzed by real-time computational algorithms such as Fast Fourier Transforms (FFT) to determine various statistics associated with EEG, including but not limited to spectral amplitudes of frequencies comprising said EEG. The statistics may be used to modify parameters of a neuromodulation signal for the purposes of optimizing therapeutic benefit. In a preferred embodiment, EEG data collected during a therapeutic neuromodulation signal application is analyzed for spectral components using an FFT algorithm. A comparison between the frequency of a stimulation signal and the highest spectral amplitude of measured EEG signal is made. If said comparison finds these frequencies to be the same, then a corresponding modification to the neuromodulation signal's frequency would be made.

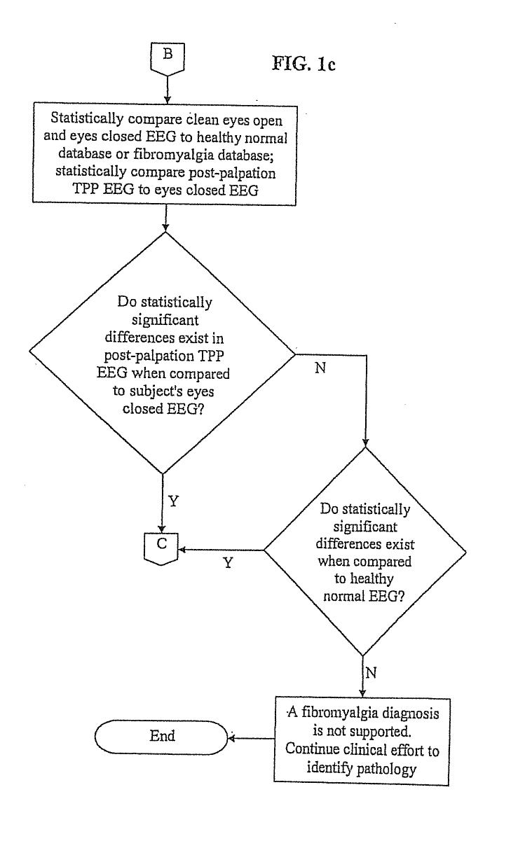

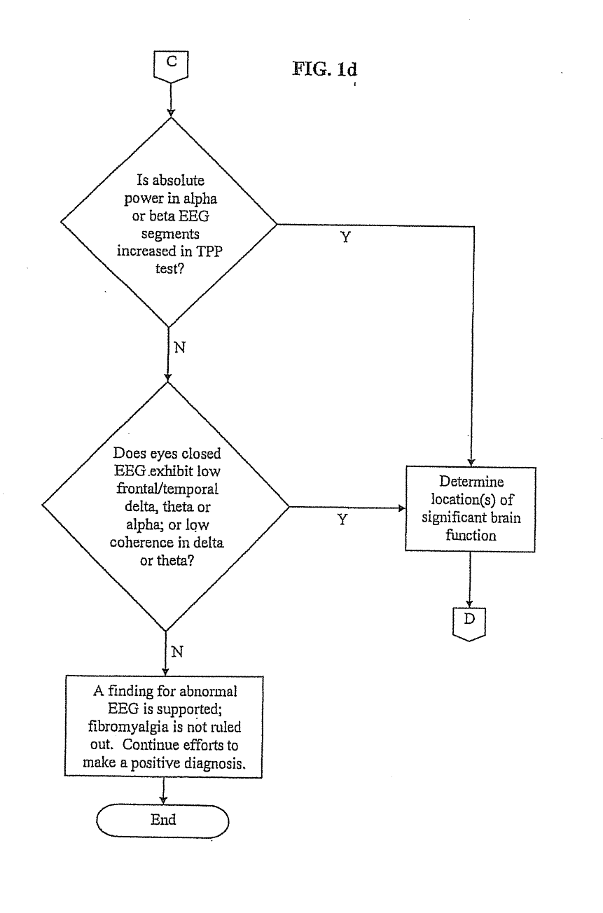

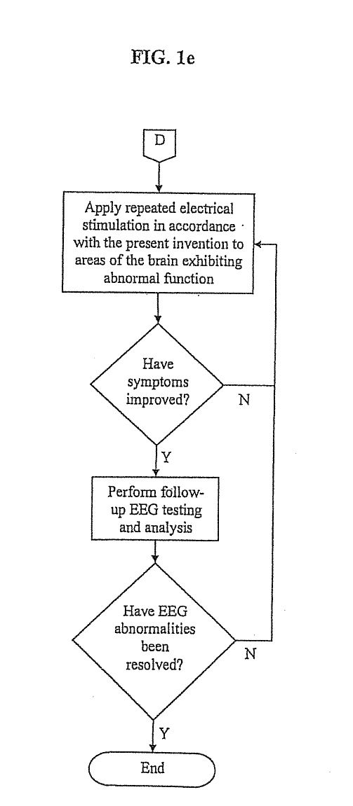

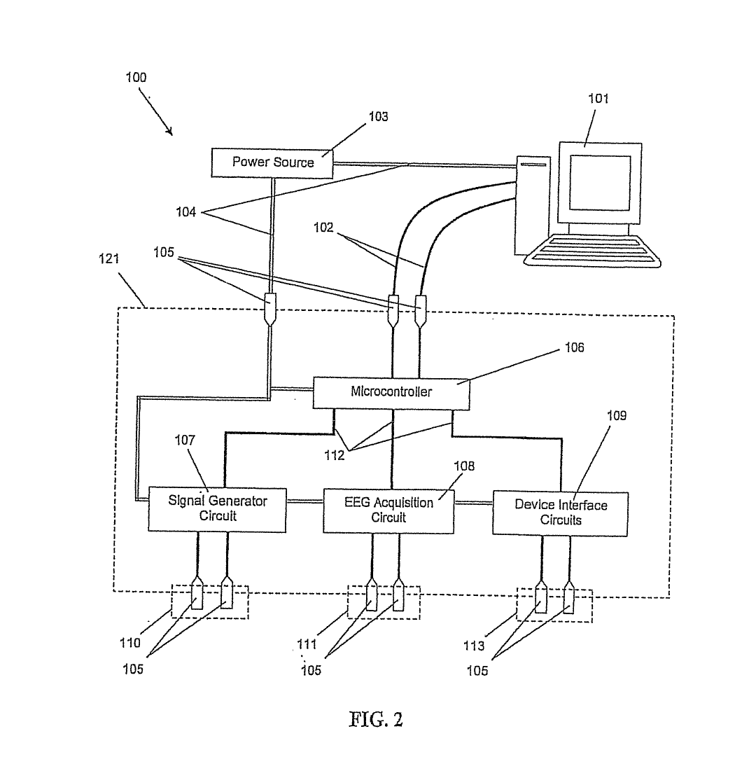

As shown in FIG. 2, the abnormal brain function diagnostic and treatment apparatus 100 may include a computer 101 interfaced to a signal generation and interface module 121 utilizing any number of methods known in the art such as the use of a computer interface cable 102. Any power source 103 known in the art to sufficiently provide power to computers and electronic devices may be utilized and externally interfaced with power wires 104. The signal generation and interface module 121 may include a microcontroller 106 electronically coupled to a signal generator circuit 107, to an EEG acquisition circuit 108 and to any number of device interface circuits 109. All external interfaces may utilize connectors 105 commonly known in the art. All electrical and electronic coupling methods may utilize conductors 112 known in the art.

In practice, the computer 101 may be configured to communicate via interface to the microcontroller 106 for various purposes including the transfer of AMPWM signal parameters and the receipt of EEG data. The computer 101 may include a user interface that allows an operator to monitor and/or influence operation of the diagnostic and treatment apparatus 100.

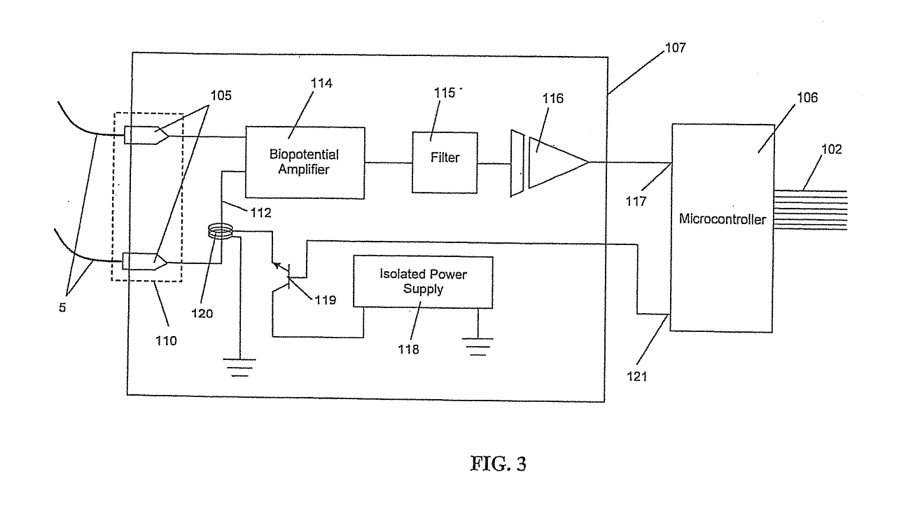

A neuromodulation signal such as an AMPWM signal may be generated in the signal generator circuit 107 and delivered to a stimulation signal interface 110 that includes connectors 105. As shown in FIG. 3, the signal generator circuit 107 may comprise a biopotential amplifier 114 that measures EEG signals and may be operatively coupled to any number of filter 115 circuits configured to reduce extraneous electrical noise in an EEG signal. The biopotential amplifier 114 may be further operatively coupled to an isolation amplifier 116 for human subject protection, and to a microcontroller 106 through an analog-to-digital interface 117. In operation, EEG may be acquired through a stimulation signal interface comprising electrical conductors 5 interfaced at connectors 105. The acquired EEG may be conducted to a biopotential amplifier 114, filter circuits 115, isolation amplifier 116 and to a microcontroller 106 for use such as, but not limited to, in software executed by an interfaced computer 101 for generating and delivering an AMPWM signal. The signal generator circuit 107 may further comprise an isolated power supply 118 configured to provide circuit power and provide human subject protection, a switching transistor 119 that has base connection to a digital-to-analog interface 122 on a microcontroller 106, and an inductor 120 configured and positioned to induce an electrical stimulation signal such as an AMPWM signal into a conductor 112 leading to a connector 105 and electrical conductor 5 in a stimulation signal interface. In operation, the microcontroller 106 may generate a stimulation signal and conduct that signal via a digital-to-analog interface 122 to the base of the switching transistor 119. Electrical power from an isolated power supply 118 may then switched on and off through the switching transistor 119 creating an amplified stimulation signal in accordance with the stimulation signal waveform generated by the microcontroller 106. The amplified stimulation signal may be further conducted to an inductor 120, and further induced into a conductor 112 creating a therapy stimulation signal in the conductor 112. The therapy stimulation signal may then be delivered to a human subject via the conductor 112 to a stimulation signal interface comprising electrical conductors 5 interfaced at connectors 105. In other words, the neuromodulation signal may be applied using an apparatus comprising a microcontroller 106 configured to generate signal waveforms and coupled to a signal generator circuit 107 configured to transform the signal waveforms into desired AMPWM neuromodulation signals. The signal generator circuit 107 may comprise circuit elements such as a biopotential amplifier 114 configured to measure EEG signals, a filter circuit 115 configured to reduce electrical noise in EEG signals, an isolation amplifier 116 configured to protect human subjects, an analog-to-digital interface 117 configured to convert analog EEG signals to digital signals, an isolated power supply 118 configured to provide circuit power and human subject protection, a switching transistor 119 configured to generate an amplified stimulation signal by switching on and off electrical power from the isolated power supply in response to stimulation signals received at a base of the switching transistor from the microcontroller, and an inductor 120 configured to induce an electrical stimulation signal into a conductor 112. Additional forms of an AMPWM signal and apparatus for generating an AMPWM signal are disclosed in the applicant's U.S. patent application Ser. No. 11/490,255 (issued as U.S. Pat. No. 7,715,910), which is incorporated herein by reference in its entirety.

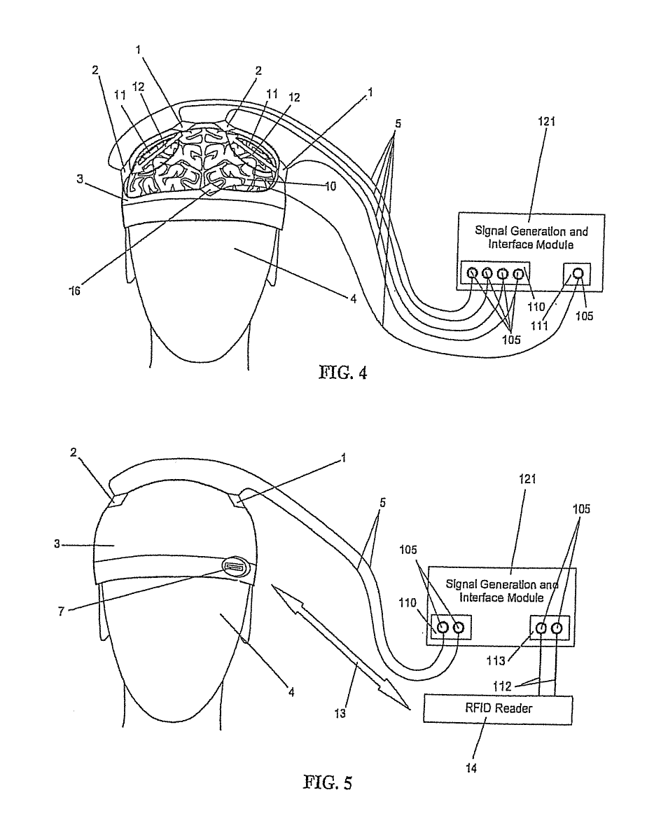

As shown in FIG. 4, the apparatus 100 may include a cap 3 configured to be worn on a subject's head in a predetermined orientation. At least two electrodes 1, 2, which may be non-invasive type electrodes, may be carried by the cap. One of the electrodes 1 may be configured to act as a stimulating electrode 1 for delivering a neuromodulation signal to the subject's head 4, and the other of the electrodes 2 may be configured to act as a ground electrode and to receive neuromodulation signals transmitted by the signal delivery electrode 1. The cap 3 may be of any suitable configuration to include a skull-cap configuration as shown in the drawings, or may simply comprise flexible bands. In any case, the cap 3 is adapted to carry the electrodes 1, 2 and to be worn on the head 4 during mitigation of abnormal brain activity, and, more particularly, to facilitate non-invasive neuromodulation signal delivery to a subject's brain.

EEG from a subject may be collected through the EEG acquisition circuit 108, which may include any form of EEG amplifier instrument known in the art, through an EEG interface 111 that may include connectors 105. At least one additional electrode 16 may be carried by the cap 3 and positioned to sense and transmit EEG signals to the EEG acquisition circuit 108. Alternatively, a stimulating electrode 1 may also serve as an EEG sensor. In other words, one or more stimulating electrodes 1 may be coupled to the EEG acquisition circuit 108 and configured to sense and transmit EEG signals to the EEG acquisition circuit 108. The cap 3 may also carry electrical conductors 5 that provide signal paths between an electrical stimulation signal source such as the signal generator circuit 107 of the diagnostic and treatment apparatus 100 and stimulating electrodes 1 and ground electrodes 2; and the electrical conductors 5 may also provide signal paths between an EEG acquisition circuit 108 and additional electrodes 16, whereby the conductors may electrically couple to connectors 105 at a stimulation signal interface 110 and an EEG interface 111 of a diagnostic and treatment apparatus 100. The stimulating electrodes 1 and ground electrodes 2 may be permanently or removably affixed into the cap 3 in cap locations where, when the cap 3 is placed on a subject's head 4 in a predetermined orientation, the stimulating electrodes 1 and ground electrodes 2 are positioned proximate to respective areas 11 of brain tissues to be stimulated, e.g., areas of brain tissue associated with abnormal brain activity.

The electrodes 1, 2 may be permanently or removably supported in cap locations on the cap 3 so that, when the cap is worn on a subject's head in a predetermined orientation, a vector path 12 extending between the stimulating electrode 1 and the ground electrode 2 passes through the desired area 11 of brain tissues to be stimulated. Further, the cap 3 may be sized in various ways to fit or to be adjustable to a variety of sizes and shapes of human heads 4 and to carry any number of stimulating electrodes 1 and ground electrodes 2 in cap locations that will cause neuromodulation signals to pass along vector paths 12 through predetermined locations of abnormal brain activity 11 in a subject's brain 10. The electrodes 1, 2 may subsequently be removed and placed in new cap locations that will cause neuromodulation signals to pass along vector paths 12 through predetermined locations of abnormal brain activity 11 in a second subject's brain 10.

The cap 3 may be configured to carry any number of electrical circuits known in the art for storing information. As shown in FIG. 5, such circuit may include a radio frequency identification (RFID) chip 7 incorporated into a cap 3 and utilized to store information including, but not limited to, the identification of the subject the cap is intended to be used on, dates and times of use, parameters of an electrical stimulation signal to be used in association with the cap and delivery of non-invasive neuromodulation, a total number of times the cap has been used and monitoring data associated with quality of use.

The diagnostic and treatment apparatus 100 may include any number of external devices that may be utilized in the process of providing assessment, diagnostics, or therapy and that may be coupled to the device interface circuit 109 and interfaced through a device interface 113 that may include connectors 105. For example, the apparatus 100 may include an RFID reader 14 for establishing electrical connectivity between the microcontroller 106 and the RFID chip 7 through a device interface 113. The use of an RFID reader 14 and RFID chip 7 creates a radio frequency pathway 13 that allows information incorporated into the RFID chip 7 to be accessed and utilized by software executed by the microcontroller 106 and/or an interfaced computer 101.

As shown in the embodiment illustrated in FIG. 6, as an alternative to the use of an RFID chip 7, other suitable methods known in the art for accessing information stored in electrical circuits may be used, such as methods that use direct electrical connections via conductors such as wires 8. Such methods may include the use of any number of programmable memory circuits 9 connected via wires 8 to a memory circuit programmer 15, which may be further interfaced to the microcontroller 106 and/or the computer 101 through the device interface 113, such that information incorporated into the programmable memory circuit 9 may be accessed and utilized by the microcontroller 106 and/or the computer 101 via, for example, software executed by the computer 101.

Alternatively, neuromodulation may be used as a method of treatment for fibromyalgia in combination with treatment of other coexisting physical conditions that may or may not be associated with fibromyalgia. In addition, or alternatively, neuromodulation may be used as a method of treatment in combination with other forms of treatment utilized to affect symptoms of fibromyalgia.

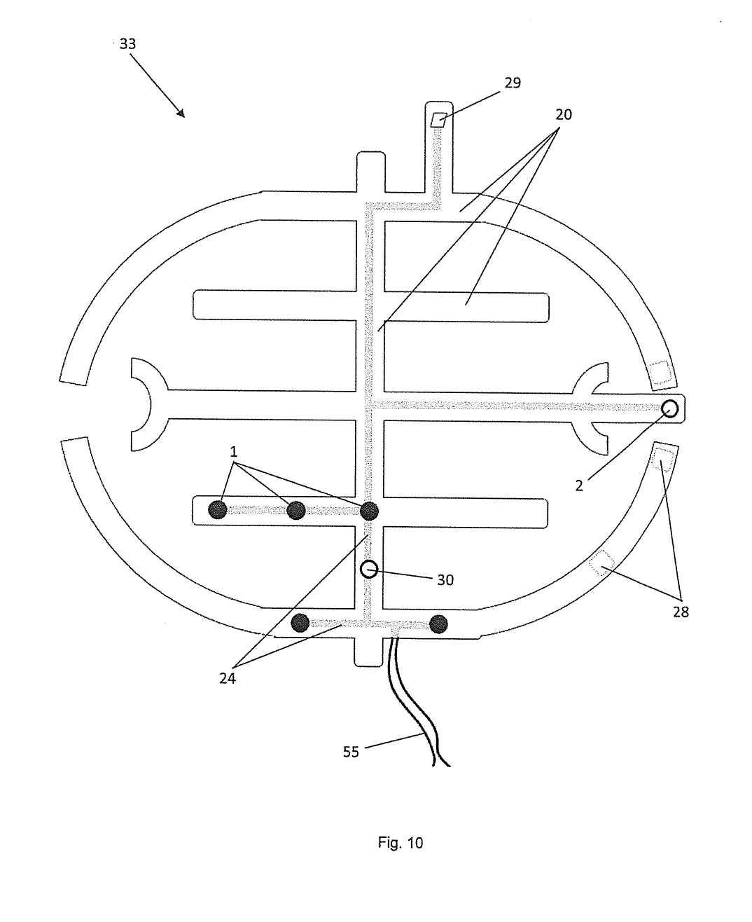

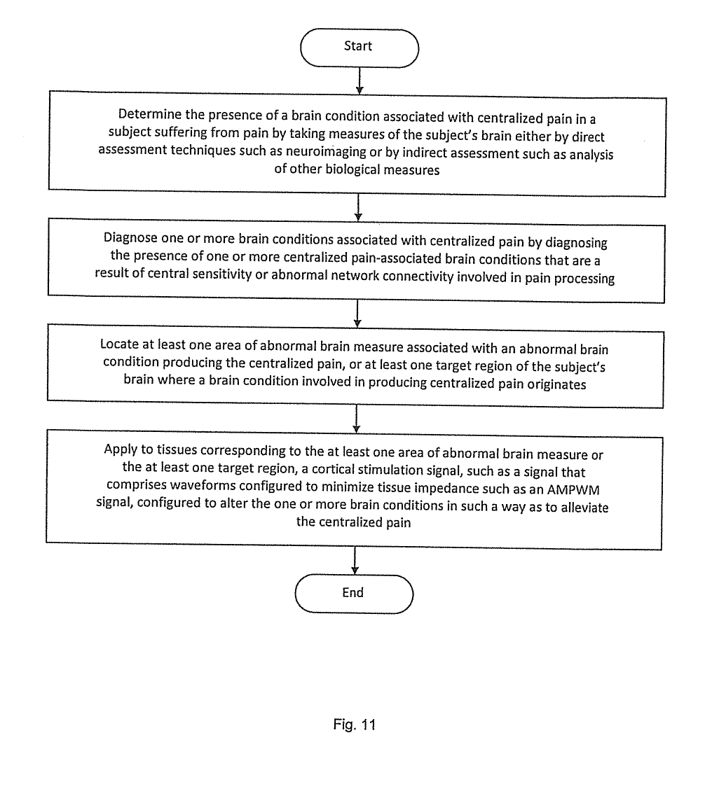

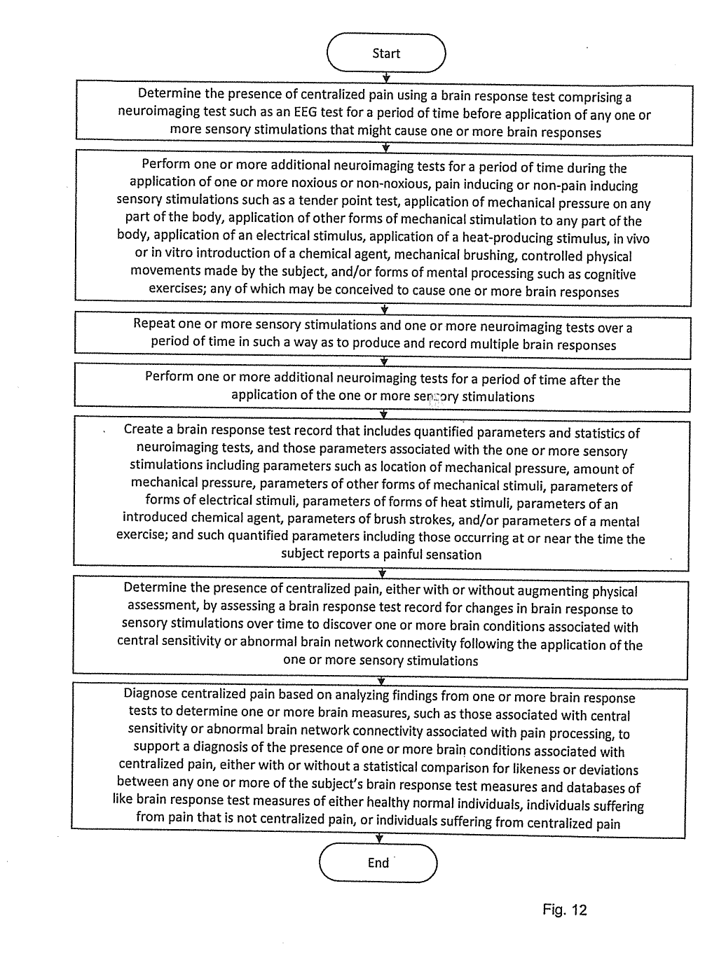

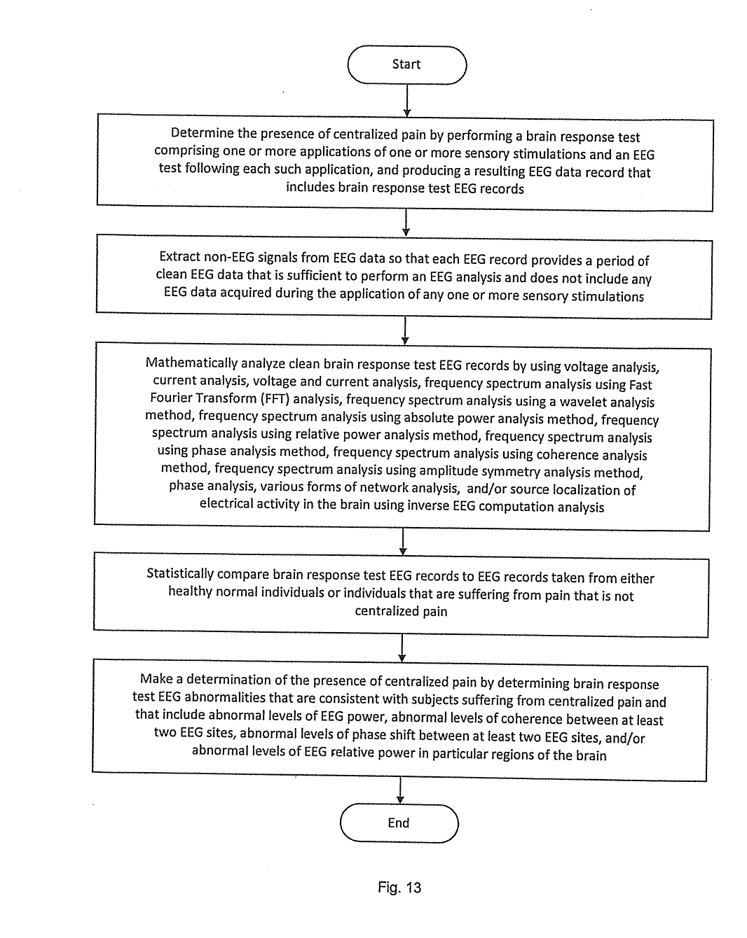

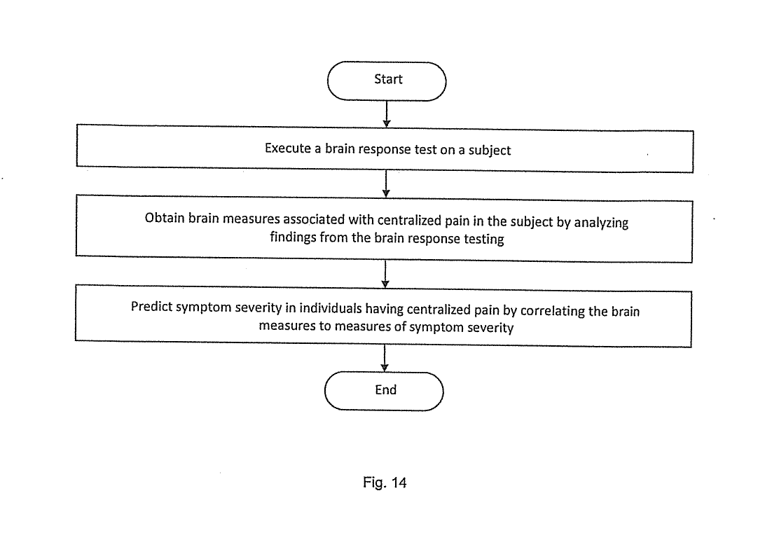

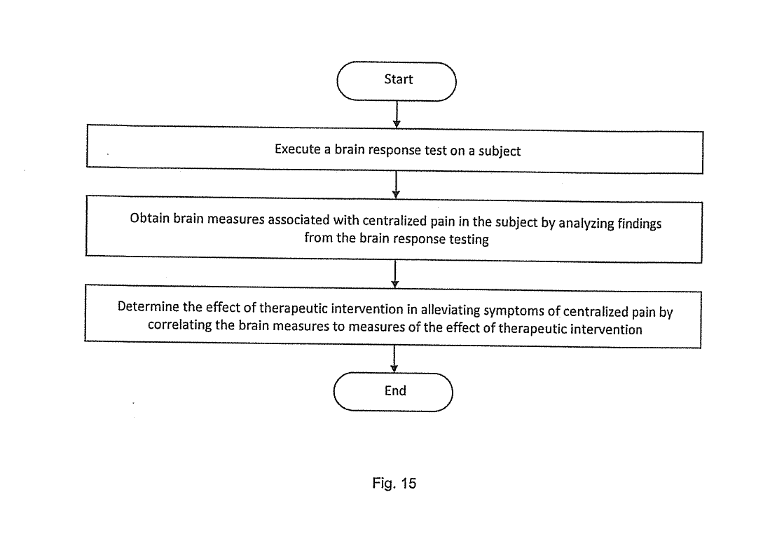

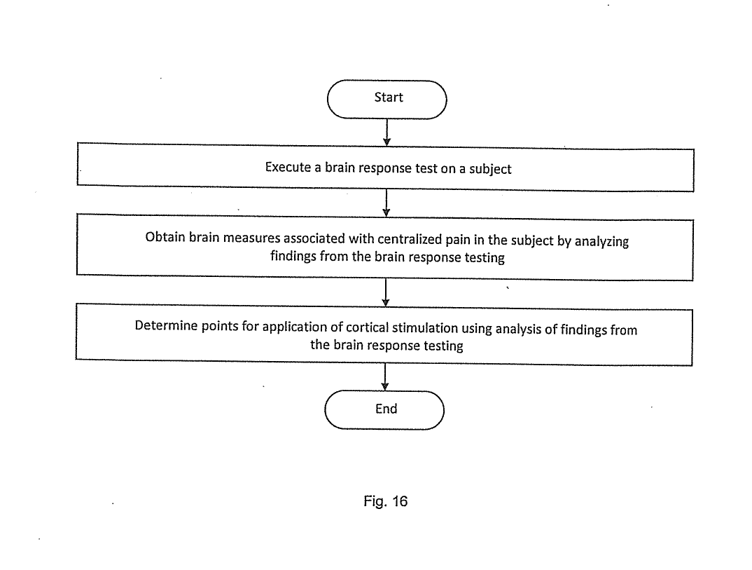

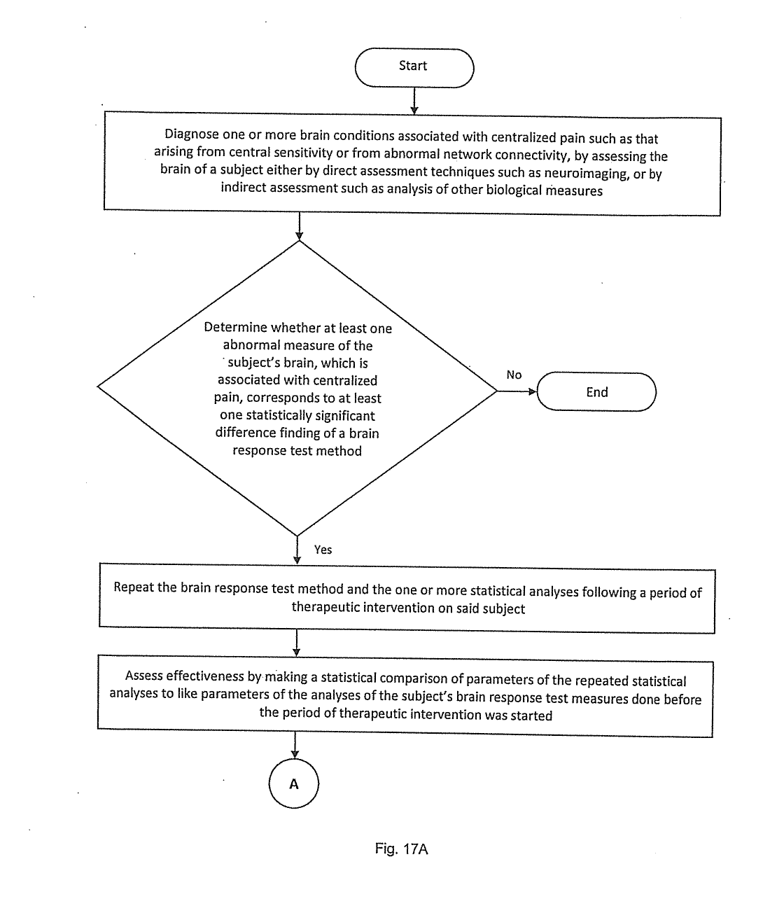

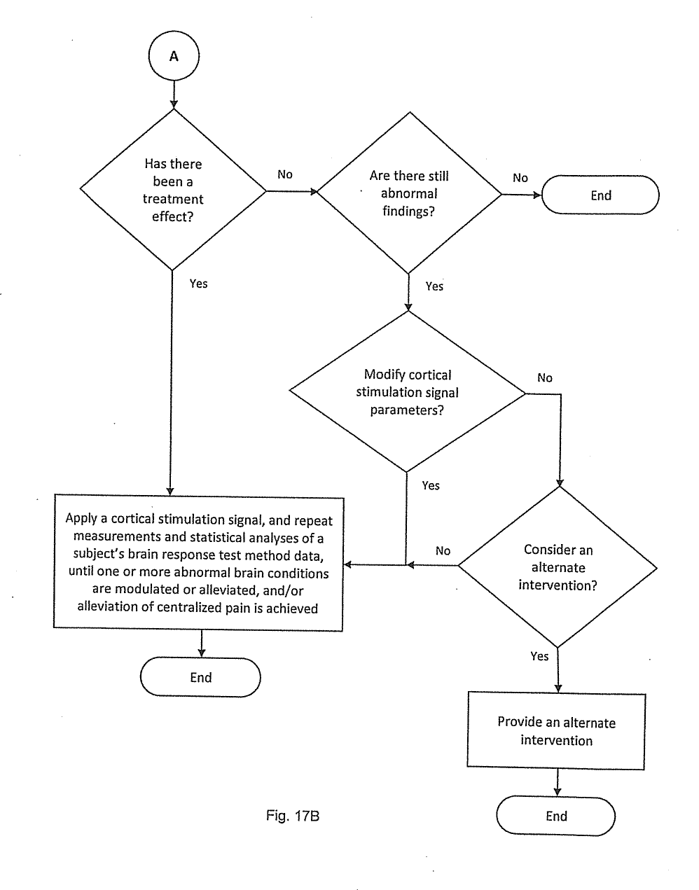

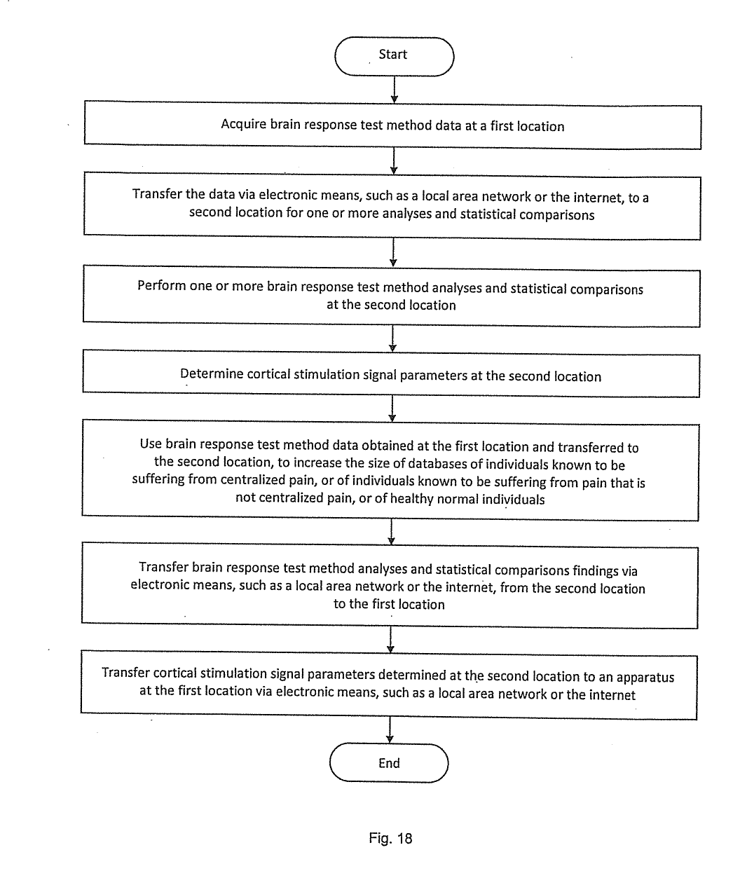

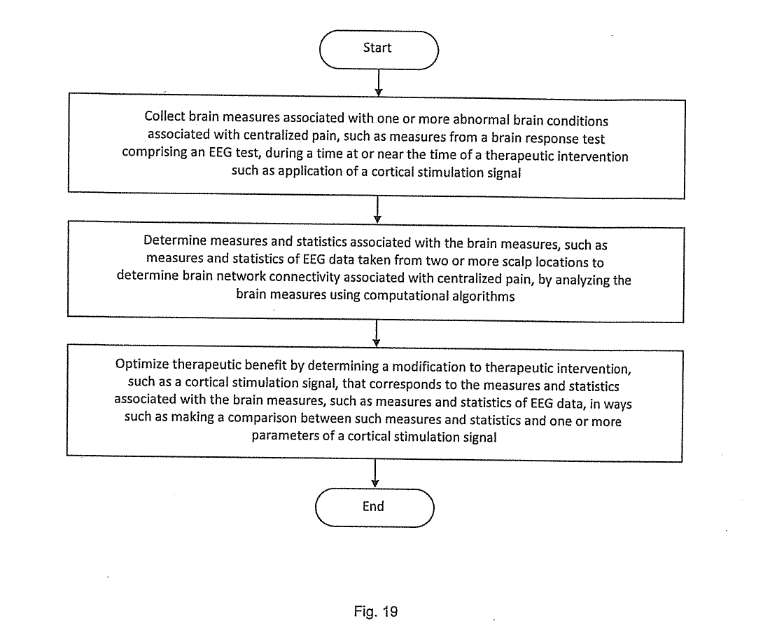

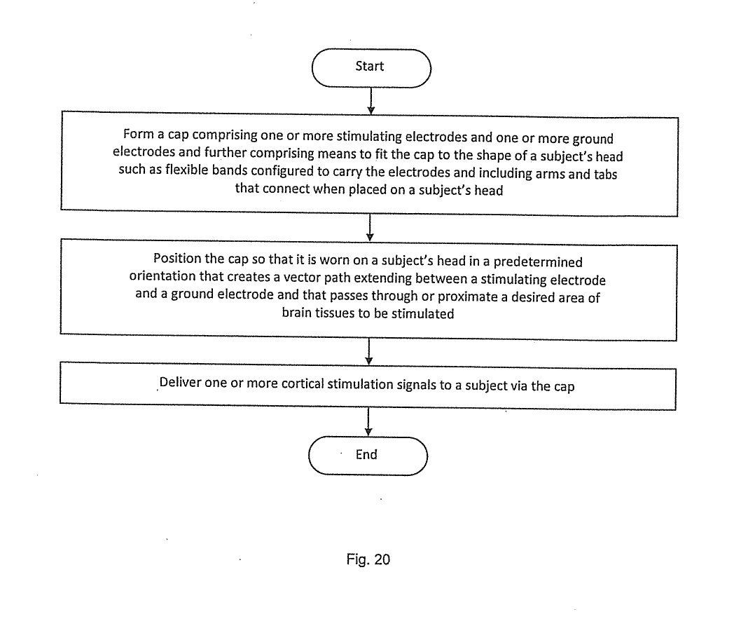

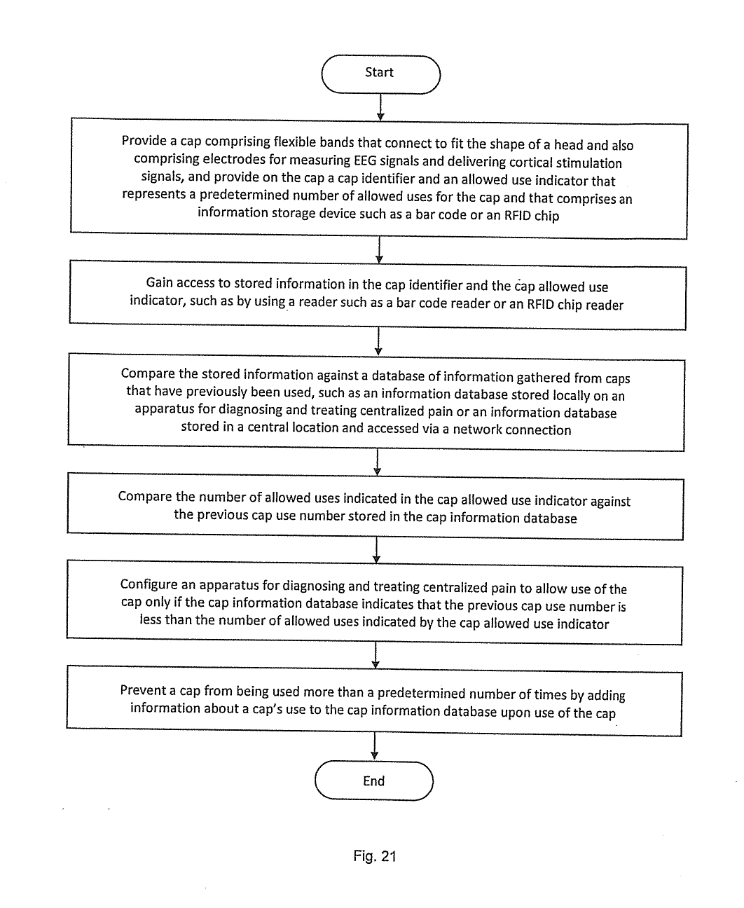

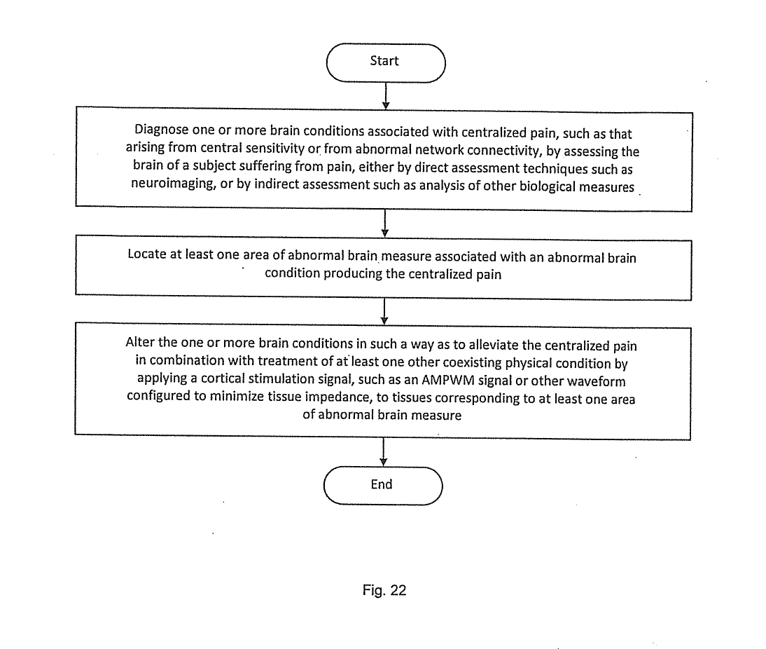

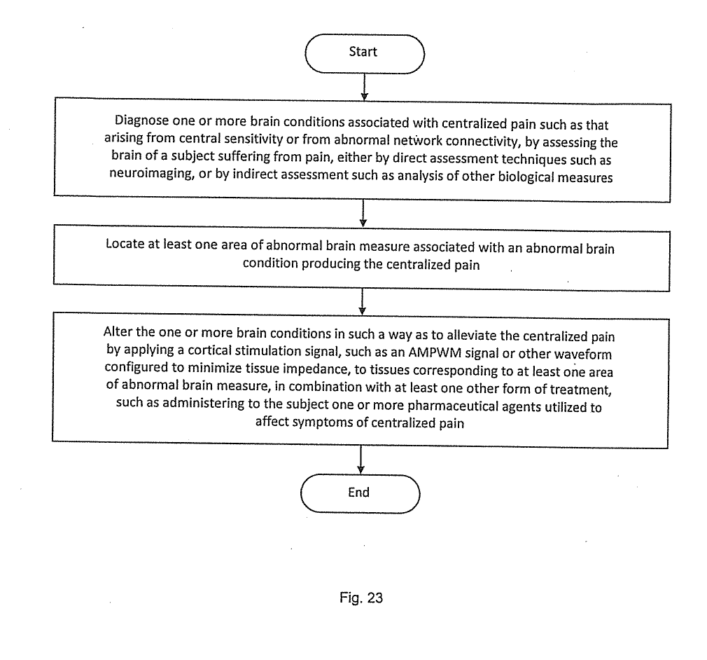

With reference to FIGS. 11-23, a method is also provided for alleviating centralized pain such as that arising from an abnormal brain condition that may include, but is not limited to, central sensitivity and abnormal network connectivity involved in pain processing. The method may include assessing the brain of a subject suffering from pain, diagnosing abnormal brain conditions associated with centralized pain, locating at least one area of abnormal brain measure associated with an abnormal brain condition producing the centralized pain and alleviating the centralized pain by applying cortical stimulation to alter the abnormal brain condition. The step of applying cortical stimulation includes, but is not limited to, the application of a cortical stimulation signal to tissues corresponding to the at least one area of abnormal brain measure. Preferably, the cortical stimulation signal comprises waveforms configured to minimize tissue impedance while effecting noninvasive cortical stimulation. Treatment effect is realized when one or more abnormal brain conditions corresponding to the at least one area of abnormal brain measure and associated with or producing the centralized pain, have been alleviated.

The step of assessing the brain of a subject suffering from pain includes, but is not limited to, making measures of a brain condition, e.g., of a brain function, brain activities or brain anatomy, either by direct assessment techniques known in the art such as neuroimaging, or by indirect assessment such as analysis of other biological measures. The assessment step includes use of any method known in the art to determine the presence of centralized pain in a subject, including methods known to identify abnormal brain conditions associated with centralized pain including, but not limited to, central sensitivity or abnormal levels of network connectivity. The diagnosing step may include making measures to support a determination of the presence of one or more brain conditions associated with centralized pain in a subject, including, but not limited to, central sensitivity or abnormal levels of network connectivity. The locating step may include making measures to identify at least one target region of the subject's brain that may be involved in centralized pain in a subject, including, but not limited to, regions involved in central sensitivity or exhibiting abnormal levels of network connectivity, or regions where abnormal brain conditions associated with centralized pain, such as central sensitivity or abnormal levels of network connectivity, originate. One skilled in the art of medical assessment may administer and interpret one or more assessments designed to detect centralized pain. Such assessments may include any one or more known neuroimaging tests. Such assessments may also be used for detecting the presence and identifying the location of one or more abnormal brain conditions through interpretation.

In a preferred embodiment, a means of assessing a brain to determine the presence of centralized pain in a subject includes the use of one or more neuroimaging tests utilizing methods and apparatuses known in the art, with the neuroimaging test being performed before, during and after the application of any one or more forms of sensory stimulation (SS) intended to cause a brain response to the SS. This test is henceforth referred to as a “brain response test” (BRT), and the SS includes any noxious, pain inducing or non-painful means. In a preferred embodiment, a BRT may include an electroencephalogram (EEG) test performed with eyes closed or eyes open, with at least one additional EEG record made that includes EEG data obtained during and after the application of any one or more forms of an SS.

One embodiment of an SS is palpation of tender points on the subject's body, consistent with the method described herein as a tender point test. Other means of causing a painful or noxious SS for the purposes of a BRT may include, but are not limited to, application of mechanical pressure on any part of the body, application of other forms of mechanical stimulation to any part of the body (e.g. a “pinch”), application of an electrical stimulus, application of a heat-producing stimulus, and in vivo or in vitro introduction of a chemical agent meant to elicit a painful or non-painful response. Means of causing a non-painful SS for the purposes of a BRT may include, but are not limited to, forms of typically non-painful physical contact including mechanical brushing, controlled physical movements made by the subject, and various forms of mental processing such as cognitive exercises.

Further to the application of an SS, the method includes any number of applications of stimulation to elicit any number of brain responses. For example, a single SS may be applied to produce a single brain response. Alternately, a series of SS applications may be made over a period of time to produce multiple brain responses so that a BRT may include assessing changes in brain response over time. Such series of PS applications may include one or more applications of any combination of noxious, painful or non-painful stimuli, with a period of rest between each application ranging from one second to several minutes. Such assessment of changes in brain response may include, but are not limited to, quantification of temporal summation of pain, also known in the pain literature as “wind up”.

The BRT test may be executed by acquiring a brain response record using any means of neuroimaging test. In a preferred embodiment, a brain response test EEG (“BRT EEG”) record is obtained that includes EEG data obtained for a period of time before, during, and after the application of any number of an SS. EEG data may be obtained from EEG electrode sites for a period of time, preferably ranging from one second to 15 minutes, prior to commencement of a first SS. During application of an SS, EEG data obtained may be denoted as EEG collected during application of the SS. Data collected during application of an SS may have unwanted aspects. For example, EEG data collected during the application of an SS may also contain measurements of electromyographic signals arising from muscle contractions a patient may make as a result of feeling a sensation such as pain. Accordingly, the data collected during the application of an SS may or may not be removed in subsequent analysis according to the method. Further to the embodiment, EEG data may be obtained from EEG electrode sites for a period of time, preferably ranging from one to 15 minutes, after application of an SS.

Further to the BRT test method, a record is made quantifying parameters associated with the one or more SS being used. For example, if an SS involves palpation of a tender point, then the location and amount of mechanical pressure being applied at or near the time the subject reports a painful sensation may be recorded. Other examples of quantification of an SS may include, but are not limited to, the amount of pressure on any body part required to elicit pain, parameters of other forms of mechanical stimuli, parameters of forms of electrical stimuli, parameters of forms of heat stimuli, parameters of an introduced chemical agent, parameters of brush strokes and parameters of a mental exercise.

Further to the BRT test method, the recording of EEG may continue for a period of time after completion of each of the one more SS applications, with the period of time preferably being between one second and 15 minutes. The process of application of an SS and subsequent recording of EEG may be repeated until all intended applications of an SS are completed. Accordingly, the resulting EEG data record includes the BRT EEG records for all applications of SS.

The BRT EEG records may be acquired for a period of time that is sufficient to extract from each BRT EEG record a record of “clean” EEG data, that is, EEG data that have minimal non-EEG signals such as extraneous electrical noise arising from, for example, instrumentation anomalies or electromyographic movement. Preferably, a record of clean EEG data is sufficient to provide enough EEG data to perform any one of a number of EEG analyses known in the art with a sufficiently high degree of statistical confidence. More preferably, all EEG records according to the method may be individually edited to provide from each EEG record a period comprising a minimum of 60 seconds of clean EEG. With regard to the BRT test method, clean data preferably does not include any EEG data acquired during the application of an SS.

Further to the BRT test method, and in the preferred embodiment, clean EEG records may be then mathematically analyzed for various time domain and frequency domain parameters of their respective electrical signals. These analyses may include, but are not limited to voltage analysis, current analysis, voltage and current analysis, frequency spectrum analysis using Fast Fourier Transform (FFT) analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, phase analysis, various forms of network analysis and source localization of electrical activity in the brain using inverse EEG computation analysis. The purpose of such analyses is to determine the presence of one or more abnormal brain conditions, e.g., brain function, brain activity, brain anatomy or related brain measures that indicate centralized pain such as, but not limited to, central sensitivity and abnormal levels of network connectivity.

According to the BRT test method, a finding of centralized pain is made by analyzing findings from the aforementioned BRT analyses. Such findings may include, but are not limited to, a determination of a brain condition associated with central sensitivity or abnormal brain network connectivity associated with pain processing. In a preferred embodiment, BRT EEG records may be statistically compared to the same parameters determined from EEG records taken from age and gender matched databases of either healthy normal individuals or individuals that are suffering from pain that is not centralized pain. Such statistical analyses may include, but are not limited to deviations from a standard normal distribution. Findings of statistically significant abnormal deviation, or lack thereof, may then be presented in a graphical or numerical format for analysis by a competent health care professional or person of similar expertise.

EEG abnormalities consistent with subjects suffering from centralized pain may include, but are not limited to one or more of the following: (1) abnormal levels of in EEG power in spectral segments of resting EEG measures, including but not limited to, an abnormal level of EEG power across the entire resting EEG spectra; (2) abnormal levels of coherence or phase shift between at least two resting EEG sites; (3) abnormal levels of resting EEG relative power in particular regions of the brain.

Further EEG abnormalities consistent with subjects suffering from centralized pain, and drawn particularly to the EEG BRT test method, may include but are not limited to a finding of (1) statistically significant increases in EEG absolute power, particularly in the alpha and beta segments, in the parietal, occipital, and temporal areas of the brain as compared to the resting EEG record for the same subject; or (2) statistically significant increases in coherence in spectral segments of the BRT EEG record as compared to the resting EEG record for the same subject.

A determination of centralized pain may be made when physical assessment findings that support a diagnosis of centralized pain are augmented by assessing a brain following a BRT. The assessment of a BRT may include a statistical comparison between any one or more of the subject's BRT measures and a database of like BRT measures of either healthy normal individuals, individuals suffering from pain that is not centralized pain, or individuals suffering from centralized pain. Alternately, centralized pain may be diagnosed by statistically determining one or more deviations between a subject's one or more BRT measures and like BRT measures obtained from at least one healthy normal individual or at least one individual suffering from pain that is not centralized pain; then comparing the one or more deviations to like deviations detected in a sample population of subjects known to be suffering from centralized pain.

In a preferred embodiment, clean resting EEG or BRT EEG records from a subject may be mathematically analyzed for various time domain and frequency domain parameters of their electrical signals, consistent with analysis techniques already described, and then findings from these mathematical analyses may be statistically compared to like parameters taken from age and gender matched databases of either healthy normal individuals, individuals suffering from pain that is not centralized pain, or individuals known to be suffering from centralized pain. The statistical comparisons may include, but are not limited to deviations from a standard normal distribution of like EEG measures associated with members of databases of healthy normal individuals, individuals suffering from pain that is not centralized pain, or individuals known to be suffering from centralized pain. The results of those comparisons may then be presented in a graphical or numerical format for analysis by a competent health care professional or person of similar expertise for the existence of statistically significant abnormal deviations, or the lack thereof. A centralized pain diagnosis would be supported if one or more findings of either resting EEG or BRT EEG records are consistent with like findings from a database comprising individuals known to be suffering from centralized pain. More preferably, a centralized pain diagnosis would be supported if one or more findings of either resting EEG or BRT EEG records are consistent with statistical significance to like findings from a database comprising individuals known to be suffering from centralized pain.

Further according to the BRT test method, measures of an abnormal brain condition, e.g., brain function, brain activity, brain anatomy or related brain measures arising from analyses of BRT test findings from a subject may be correlated to measures of symptom severity, such as but not limited to pain severity. Such correlation has utility in both predicting symptom severity in individuals with centralized pain, and in determining the effect of therapeutic intervention to alleviate symptoms of centralized pain.

Further, BRT test analyses according to the method may also be used for determining the location of an abnormal brain condition and further for determining points for application of cortical stimulation according to the method recited herein for alleviating centralized pain.

Alleviation of centralized pain, including but not limited to central sensitivity and abnormal brain network connectivity associated with centralized pain, may include the application of various forms of cortical stimulation in a manner designed to alleviate the abnormal brain condition identified in accordance with the aforementioned BRT test method and analyses. Although any form of cortical stimulation is included in the method, a preferred embodiment includes noninvasive neuromodulation techniques, and more preferably includes use of an AMPWM signal form as disclosed in applicant's U.S. patent application Ser. No. 11/490,255, which has issued as U.S. Pat. No. 7,715,910 and which is attached as Appendix A, and applicant's International Patent Application Ser. No. PCT/US2008/72395 all of which are incorporated herein by reference.

Alleviation of one or more abnormal brain conditions associated with centralized pain may be accomplished by generating and applying to the subject a cortical stimulation signal having at least one parameter configured to modulate at least one abnormal brain measure that is associated with one or more abnormal brain conditions associated with centralized pain, and that corresponds to at least one statistically significant difference finding of a BRT test method. Although any form of cortical stimulation is included in the method, a preferred embodiment includes noninvasive neuromodulation techniques, and more preferably includes use of an AMPWM signal form as disclosed in applicant's U.S. Pat. No. 7,715,910, which is attached as Appendix A, and applicant's International Patent Application Ser. No. PCT/US2008/72395, which are incorporated herein by reference.

The method may include the steps of repeating one or more analyses in accordance with the BRT test method, as described herein, following a period of therapeutic intervention on a subject. The method may further comprise statistical comparison of parameters of the repeated analyses to like parameters of the analyses of the subject's BRT measures done before the period of therapeutic intervention was started. These comparisons may be used to assess the effectiveness of the therapeutic intervention, preferably cortical stimulation, or to determine if an alternate intervention is indicated in the absence of treatment effect from a current therapeutic intervention. The comparisons could also be used to determine if further therapeutic intervention is indicated in the absence of any abnormal findings. The comparisons may further be used to modify cortical stimulation signal parameters, preferably AMPWM signal parameters, in accordance with the findings of the repeated quantitative assessment step.

With specific reference to the BRT test method, repeat testing may include the application of one or more SS forms. The application of the one or more SS forms may be done in accordance with types and levels quantified for the same form of SS that was used or performed before therapeutic intervention.

Further according to the method, BRT test method data may be acquired at a first location (e.g. a clinical location) and the acquired BRT test method data transferred via electronic means to another location (e.g. a central analysis location) for the herein described analysis and statistical comparisons to be accomplished. The electronic means of data transfer may include, but isn't limited to means of data transfer across a local area network and/or the internet. Consequently, analysis and statistical findings may then be transferred from a central analysis location to a clinical location, where they may be used in various ways by a physician or similarly qualified health care professional for the determination of parameters of a cortical stimulation signal used for therapeutic intervention, treatment or alleviation of centralized pain.

Further according to the method, BRT test method data may be acquired at a first location (e.g. a clinical location) and the acquired BRT test method data transferred via electronic means to another location (e.g. a central analysis location) for a purpose such as inclusion or increasing the size of various databases of individuals known to be suffering from centralized pain, individuals known to be suffering from pain that is not centralized pain, and healthy normal individuals.

Further according to the method, cortical stimulation signal parameters may be determined at a central analysis location and subsequently transferred as data via electronic means to an apparatus at another location (e.g. a clinical location) provided for delivery of a cortical stimulation signal used for therapeutic intervention, treatment or alleviation of centralized pain. The electronic means of data transfer may include, but isn't limited to, means of data transfer across a local area network and/or the internet.

The steps of application of the cortical stimulation signal and repeat measurements and analyses of a subject's BRT test method data may be continued until one or more abnormal brain conditions as determined by, for example, measures from further BRT test method analysis, are modulated or alleviated, and/or alleviation of centralized pain is achieved.

Further according to the method, measures associated one or more brain conditions associated with centralized pain may be collected during a time period at or near the time of a therapeutic intervention such as application of a cortical stimulation signal, and the measures may be analyzed by real-time computational algorithms to determine various measures and statistics associated with the brain conditions associated with centralized pain. The measures and statistics may then be used to modify parameters of an intervention for the purposes of optimizing therapeutic benefit. In a preferred embodiment, EEG data from one or more scalp locations are collected during a time period at or near the time of a therapeutic intervention such as application of a cortical stimulation signal, and an FFT is performed on the EEG data to determine various measures and statistics comprising spectral parameters of the EEG data including, but not limited to, spectral amplitudes of said EEG data. In another preferred embodiment, EEG data from two or more scalp locations are analyzed to determine measures and statistics of brain network connectivity associated with centralized pain. A comparison between any number of parameters of a cortical stimulation signal and the measures and statistics according to the embodiment can be made to determine a corresponding modification to the cortical stimulation signal's parameters for the purpose of optimizing therapeutic benefit.

The application of a cortical stimulation signal is preferably accomplished with an AMPWM signal, and may comprise the steps of choosing AMPWM signal parameters intended to alleviate one or more abnormal brain conditions associated with centralized pain identified in accordance with the BRT test method, generating the AMPWM signal, and applying the AMPWM signal to a subject using any of the various apparatuses for AMPWM signal generation and delivery disclosed herein. Delivery of the AMPWM signal may be accomplished by utilizing an electrode set comprising one or more non-invasive stimulating electrodes adapted to be worn by a subject, as also disclosed herein.

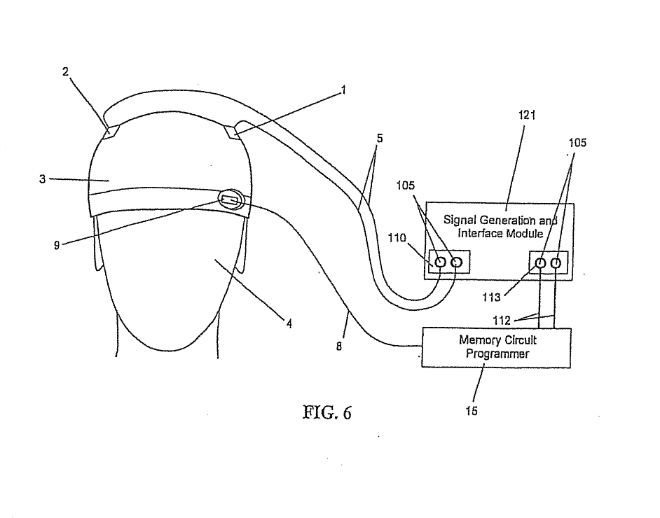

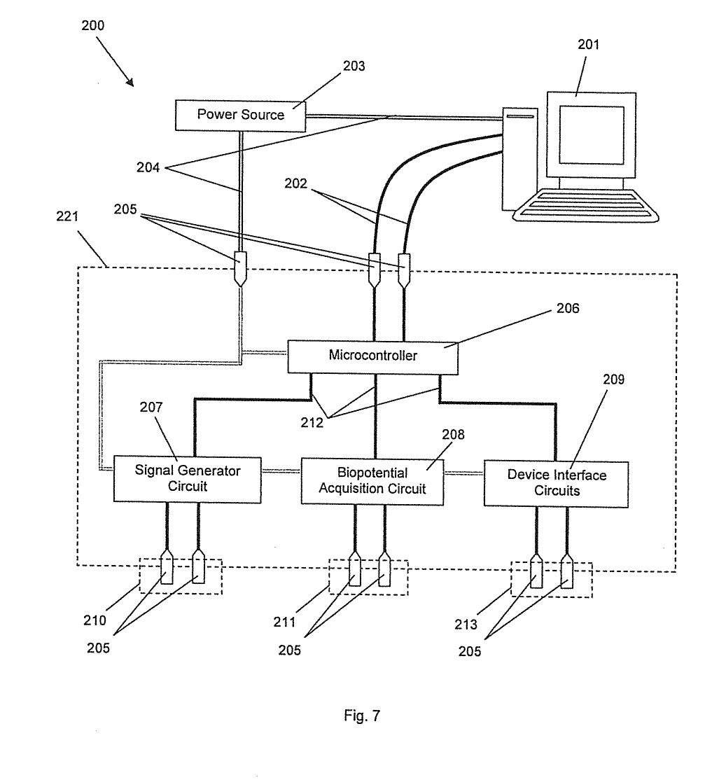

In addition, and as shown in FIG. 7, an apparatus 200 for diagnosing and treating centralized pain may include a computer 201 interfaced to a signal generation and interface module 221 utilizing any number of methods known in the art such as the use of a computer interface cable 202. Any power source 203 known in the art to sufficiently provide power to computers and electronic devices may be utilized and externally interfaced with power wires 204. The signal generation and interface module 221 may include a microcontroller 206 electronically coupled to a signal generator circuit 207, to a biopotential acquisition circuit 208, such as an EEG acquisition circuit, and to any number of device interface circuits 209. All external interfaces may utilize connectors 205 commonly known in the art. All electrical and electronic coupling methods may utilize conductors 212 known in the art.

In practice, the computer 201 may be configured to communicate via interface 202 to the microcontroller 206 for various purposes including the transfer of cortical stimulation signal parameters and the receipt of biopotential data. The computer 201 may include a user interface that allows an operator to monitor and/or influence operation of the apparatus 200 for diagnosing and treating centralized pain.

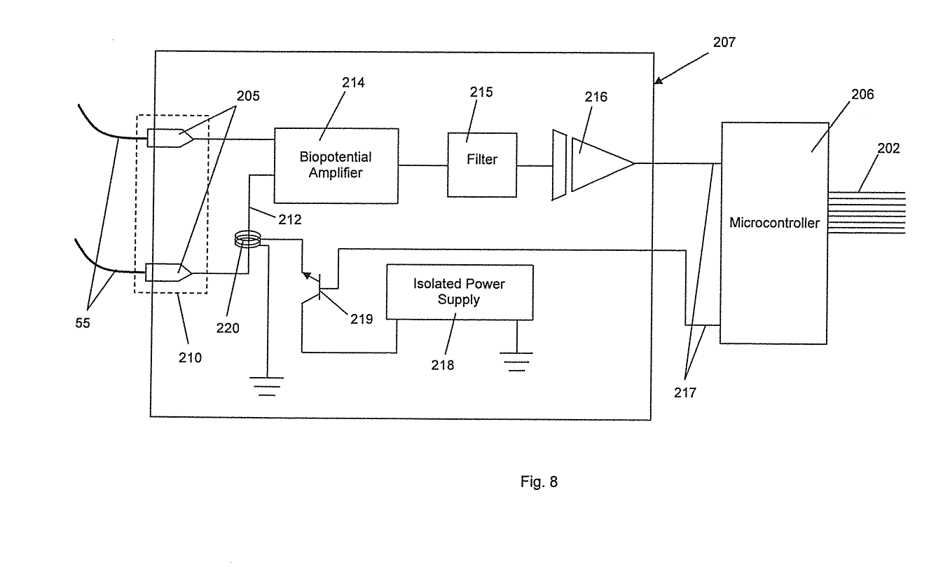

A cortical stimulation signal such as an AMPWM signal may be generated in the signal generator circuit 207 and delivered to a stimulation signal interface 210 that includes connectors 205. As shown in FIG. 8, the signal generator circuit 207 may comprise a biopotential amplifier 214, such as a biopotential amplifier configured to measure EEG signals, and may be operatively coupled to any number of filter circuits 215 configured to reduce extraneous electrical noise in a biopotential signal. The biopotential amplifier 214 may be further operatively coupled to an isolation amplifier 216 for human subject protection, and to a microcontroller 206 through an analog-to-digital interface 217. In operation, one or more biopotential signals may be acquired through a stimulation signal interface 210 comprising electrical conductors 55 interfaced at connectors 205. The acquired biopotential signal may be conducted to a biopotential amplifier 214, filter circuits 215, isolation amplifier 216 and to a microcontroller 206 for use such as, but not limited to, in software executed by an interfaced computer 201 for generating and delivering a cortical stimulation signal. The signal generator circuit 207 may further comprise an isolated power supply 218 configured to provide circuit power and provide human subject protection, a switching transistor 219 that has base connection to a digital-to-analog interface 222 on a microcontroller 206, and an inductor 220 configured and positioned to induce an electrical stimulation signal such as a cortical stimulation signal into a conductor 212 leading to a connector 205 in a stimulation signal interface 210. In operation, the microcontroller 206 may generate an electrical stimulation signal and conduct that signal via a digital-to-analog interface 222 to the base of the switching transistor 219. Electrical power from an isolated power supply 218 may then switched on and off through the switching transistor 219 creating an amplified stimulation signal in accordance with the electrical stimulation signal waveform generated by the microcontroller 206. The amplified stimulation signal may be further conducted to an inductor 220, and further induced into a conductor 212 creating a cortical stimulation signal in the conductor 212. The cortical stimulation signal may then be delivered to a human subject via the conductor 212 to a cortical stimulation signal interface 210 comprising electrical conductors 55 interfaced at connectors 205. In other words, the cortical stimulation signal may be applied using an apparatus comprising a microcontroller 206 configured to generate signal waveforms and coupled to a signal generator circuit 207 configured to transform the signal waveforms into desired cortical stimulation signals. The signal generator circuit 207 may comprise circuit elements such as a biopotential amplifier 214 configured to measure biopotential signals, a filter circuit 215 configured to reduce electrical noise in biopotential signals, an isolation amplifier 216 configured to protect human subjects, an analog-to-digital interface 217 configured to convert analog biopotential signals to digital signals, an isolated power supply 218 configured to provide circuit power and human subject protection, a switching transistor 219 configured to generate an amplified stimulation signal by switching on and off electrical power from the isolated power supply in response to electrical stimulation signals received at a base of the switching transistor from the microcontroller, and an inductor 220 configured to induce an amplified stimulation signal into a conductor 212. Additional forms of cortical stimulation signals such as an AMPWM signal and apparatus for generating an AMPWM signal are disclosed in the applicant's U.S. Pat. No. 7,715,910, which is attached as Appendix A.

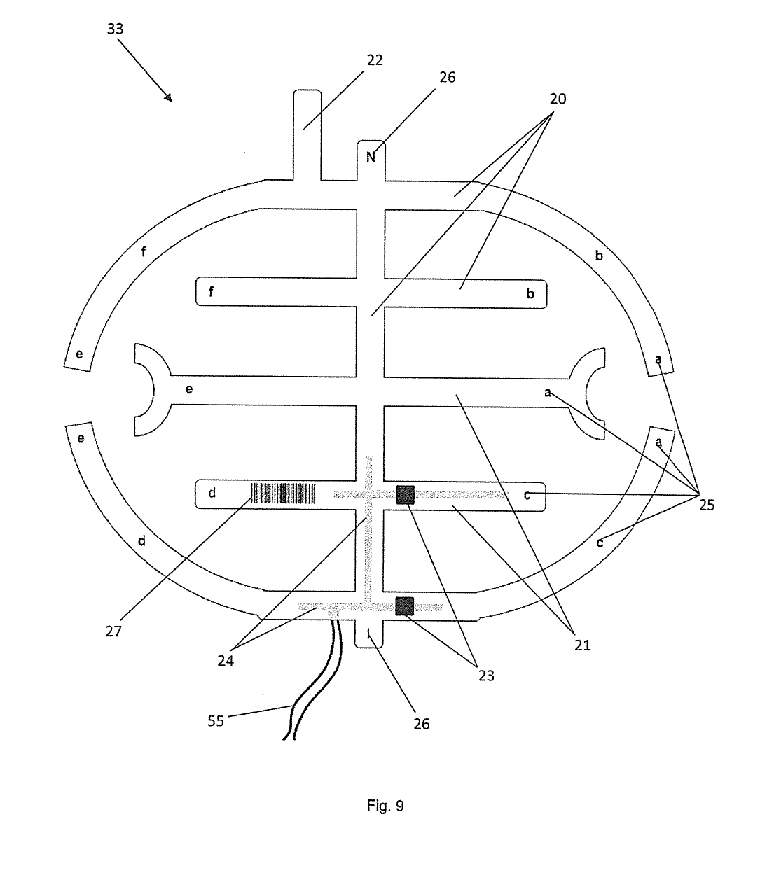

In addition, and further to a cap 3 configured to be worn on a subject's head, an alternate embodiment of such a cap is shown at 33 in FIGS. 9 and 10, which may be used with an abnormal brain function diagnostic and treatment apparatus 100 and/or an apparatus 200 for diagnosing and treating centralized pain. The cap 33 may include a series of interconnectable flexible bands 20 configured to include arms 21 and tabs 22 that connect when placed on a head to form a means to fit the shape of the head. The flexible bands 20 may comprise substrate materials further comprising electrical circuits 23 and conductive pathways 24 integral to the substrate materials. Further, the cap 33 may be adapted to carry any number of electrodes 1, 2, 30 for the purposes of both measuring one or more EEG signals and delivering one or more cortical stimulation signals to a subject for the purposes of assessing or alleviating abnormal brain measures associated with centralized pain, or altering network connectivity.

According to one embodiment, the cap 33 may comprise a series of interconnectable flexible bands 20 that may be made with materials designed to facilitate manufacturing for desired product variability. The materials may also be chosen to be inexpensive and disposable.

In one embodiment, the flexible bands 20 comprise electrically conductive pathways 24 leading to any number of electrodes 1, 2, 30 that may be integrated into the flexible bands 20. The electrodes 1, 2, 30 may be of any form suitable for either EEG measurement or conduction of a cortical stimulation signal, and may preferably be of a pre-prepared and pre-gelled form. The flexible bands 20 may further comprise areas of reusable self-adhesive material 28 to facilitate interconnection of the flexible bands 20 when placed on a head to form a cap 33 that beneficially fits the shape of the head. The flexible bands 20 may also comprise a series of markers 25 imprinted on various arms 21 and tabs 22, with the markers 25 individually conceived and located in ways to aid in proper connection and sizing to form a cap 33 on different sizes and shapes of a head. Further, the flexible bands 20 may also comprise a series of placement markers 26 imprinted on various arms 21 and tabs 22 to aid in proper placement of a cap 33 on a head. Further still, the electrically conductive pathways 24 in the flexible bands 20 may interface to electrical conductors 55 that may electrically couple to connectors 105 at a stimulation signal interface 110 and/or an EEG interface 111 of an abnormal brain function diagnostic and treatment apparatus 100 (FIG. 2 and 3); or to electrical conductors 55 that electrically couple to connectors 205 at a stimulation signal interface 210 and/or an EEG interface 211 of an apparatus 200 for diagnosing and treating centralized pain (FIGS. 7 and 8).

Further to the embodiment, the flexible bands 20 may also comprise one or more electrical circuits 23 such as integrated circuits configured to facilitate the measurement of one or more EEG signals or the delivery of one or more cortical stimulation signals, both to and from a head. Examples of such circuits may include, but are not limited to, signal conditioning circuits such as pre-amplifiers or filters, circuits for generating cortical stimulation signals, digital-to-analog converters and analog-to-digital converters.

Further to the embodiment, when interconnected the flexible bands 20 form a cap 33 that provides for electrode 1, 2, 30 placement such that individual electrodes can be used for the purposes of either (1) measuring one or more EEG signals, (2) delivering one or more cortical stimulation signals to a subject, or (3) both measuring one or more EEG signals and delivering one or more cortical stimulation signals to a subject. Electrode placement may be consistent with a standard international 10-20 electrode positioning system for full EEG measurement, or a modified 10-20 electrode positioning system having fewer electrodes than the standard international 10-20 electrode positioning system for localized or strategically limited EEG recordings. Alternately, the flexible bands 20 may be configured to place one or more electrodes 30 in locations that are not consistent with the standard international 10-20 electrode positioning system. Alternately, the number of flexible bands 20 may be limited to only provide for placement and positioning of one or more electrodes 1, 2, 30 in accordance with any positioning system, and therefore may not form a complete cap 33 when interconnected and placed on a head.

Further to the embodiment, electrodes 1, 2, 30 positioned in flexible bands 20 may be configured to both provide for EEG measurement from a subject and conduction of a cortical stimulation signal to a subject at overlapping times.

Further to the embodiment, the flexible bands 20 may also comprise arms 21 and tabs 22 that position electrodes 1, 2, 30 acting as ground electrodes to rest on the skin at non-EEG measurement sites such as, but not limited to sites on one or both of a subject's ears.

Further to the embodiment, the flexible bands 20, arms 21 or tabs 22 also comprise one or more electrical circuits 23 that further comprise one or more sensors 29 configured to provide non-EEG measures. An example of one such embodiment includes arms 21, tabs 22, electrical circuits 23 and sensors 29 configured to measure an electrooculogram for purposes including, but not limited to measurement of eye movements to aid in removal of non-EEG signals from an EEG record. Another example of an embodiment includes arms 21, tabs 22, electrical circuits 23 and sensors 29 configured to measure electromyographic activity on or near a head for purposes including, but not limited to measurement of muscle movements to aid in removal of non-EEG signals from an EEG record. Still another example of an embodiment includes arms 21, tabs 22, electrical circuits 23 and sensors 29 such as an accelerometer configured to measure physical motion of a subject for purposes including, but not limited to measurement of physical motion to aid in removal of non-EEG signals from an EEG record.

Further to the embodiment, electrodes 1, 2, 30 including one or more ground electrodes may be positioned in flexible bands 20 for delivering one or more cortical stimulation signals to a subject. The combined positioning of electrodes 1, 2, 30 may be arranged so that when the cap 33 is worn on a subject's head in a predetermined orientation, a vector path extending between a stimulating electrode 1, 30 and a ground electrode 2 passes through or proximate the desired area of brain tissues to be stimulated.

As an alternate embodiment, a cap 33 may comprise any combination of a flexible material such as cloth or paper configured to cover at least a portion of a head, flexible bands 20 made of one or more materials that do not conduct electricity, and flexible bands 20 that do conduct electricity; all configured to provide alternate ways to accomplish the various cap 33 embodiments disclosed herein.

Other embodiments of caps taught herein as being configured to be worn on a subject's head may be configured to carry any number of means known in the art for storing, transmitting or otherwise providing information. Such information may include electronic data or means of accessing electronic data, and may further include, but is not limited to, a unique identification code that indicates what subject the cap is intended or designated for use on, dates and times of use, parameters of a cortical stimulation signal to be used in association with the cap, the total number of times the cap has been used, and/or monitoring data associated with quality of use. Where it is desired that a cap be used only one time and subsequently disposed, such information also includes means of indicating that the cap has been used, and/or information used by an abnormal brain function diagnostic and treatment apparatus 100 or an apparatus 200 for diagnosing and treating centralized pain to prevent the cap from being used again.

As previously disclosed, a cap 3 may be configured to carry any number of electrical circuits known in the art for storing information. As shown in FIG. 5, such circuit may include a radio frequency identification (RFID) chip 7. As shown in FIG. 6, any number of programmable memory circuits 9 may be used as an alternative to the use of an RFID chip 7. Such methods may be practiced using any one of the disclosed cap embodiments. In a preferred embodiment, any one or more bar coding methods known in the art may be used for encoding and decoding data, whereby the bar code 27 may be inextricably integrated into any element of the cap 33. For example, a bar code 27 may be printed onto a flexible band 20 of a cap 33. Further to this embodiment, an abnormal brain function diagnostic and treatment apparatus 100 or an apparatus 200 for diagnosing and treating centralized pain may include a bar code reader (not shown) for which methods of use are also well-known in the art, and which may be used for accessing coded information stored in a bar code 27 on a cap 33 in such a way that the information incorporated into the bar code may be utilized by software executed by a microcontroller 106 and/or an interfaced computer 101 on an abnormal brain function diagnostic and treatment apparatus 100, or executed by a microcontroller 206 and/or an interfaced computer 201 on the apparatus 200 for diagnosing and treating centralized pain.

Also provided is a means of preventing a cap from being used more than an allowed number of times, where the allowed number is preferably one. For example, this may be achieved by encoding a unique identification code for the cap 33 into a bar code 27 integrated into an element of the cap 33. Alternately, a number of allowed uses may also be encoded into the cap's bar code 27. Upon use of the cap 33, the user of an abnormal brain function diagnostic and treatment apparatus 100 or an apparatus 200 for diagnosing and treating centralized pain, may use a bar code reader to access coded information stored on the bar code 27, including at least the unique identification code. Software executed by a microcontroller 106 and/or an interfaced computer 101 on an abnormal brain function diagnostic and treatment apparatus 100, or executed by a microcontroller 206 and/or an interfaced computer 201 on an apparatus 200 for diagnosing and treating centralized pain, may compare the unique identification code against a database of unique identification codes of caps 33 that have previously been used. If the number of previous uses stored in the database is less than the number of allowed uses, then the abnormal brain function diagnostic and treatment apparatus 100 or the apparatus 200 for diagnosing and treating centralized pain will permit the cap 33 to be used, that is, either apparatus 100, 200 will deliver a cortical stimulation signal to the cap 33. In this case, information about the cap's use such as the unique identification code will be added to a database of unique identification codes of caps 33. In the embodiment, the database may be stored locally on the apparatus 100, 200, and/or it may be stored in a central location and accessed via network connection such as the internet. If, on the other hand, the number of previous uses stored in the database is equal to the number of allowed uses, then the apparatus 100, 200 will not permit the cap 33 to be used, that is, the apparatus 100, 200 will not deliver a cortical stimulation signal to the cap 33.

The apparatus 200 for diagnosing and treating centralized pain may also include any number of external devices that may be utilized in the process of providing assessment, diagnostics, or therapy and that may be coupled to the device interface circuit 209 and interfaced through a device interface 213 that may include connectors 205. For example, the apparatus 200 may include a bar code reader for scanning a bar code 27 on a cap 33, or an RFID reader for reading information on an RFID chip 7 on a cap 3.

Cortical stimulation may be used as a method of alleviating centralized pain in combination with treatment of other coexisting physical conditions that may or may not be associated with centralized pain. In addition, or alternatively, cortical stimulation may be used as a method of alleviating centralized pain in combination with other forms of treatment utilized to affect symptoms of centralized pain including, but not limited to, administering one or more pharmaceutical agents to the subject to further augment alleviating centralized pain.

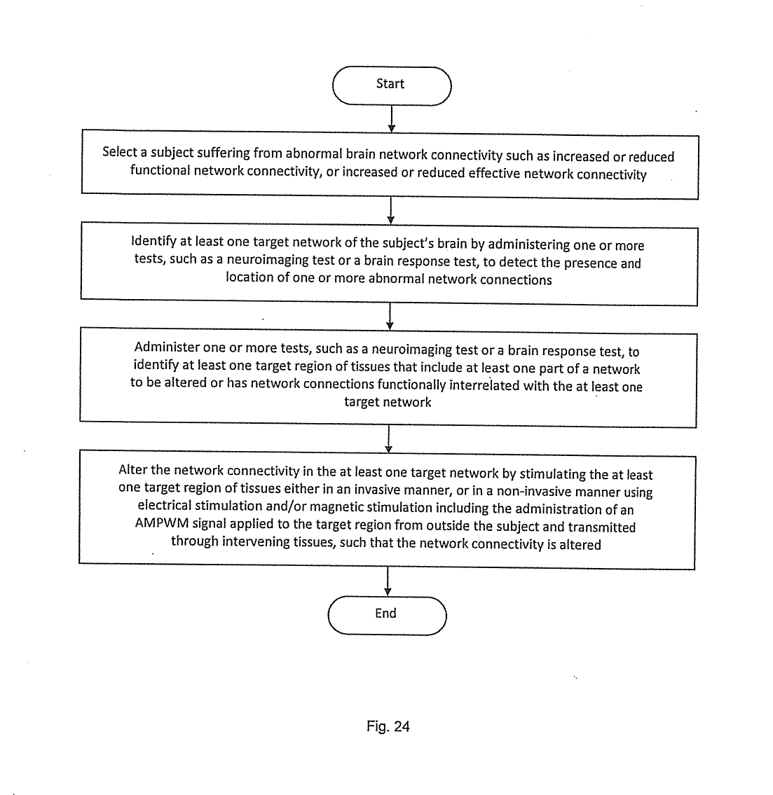

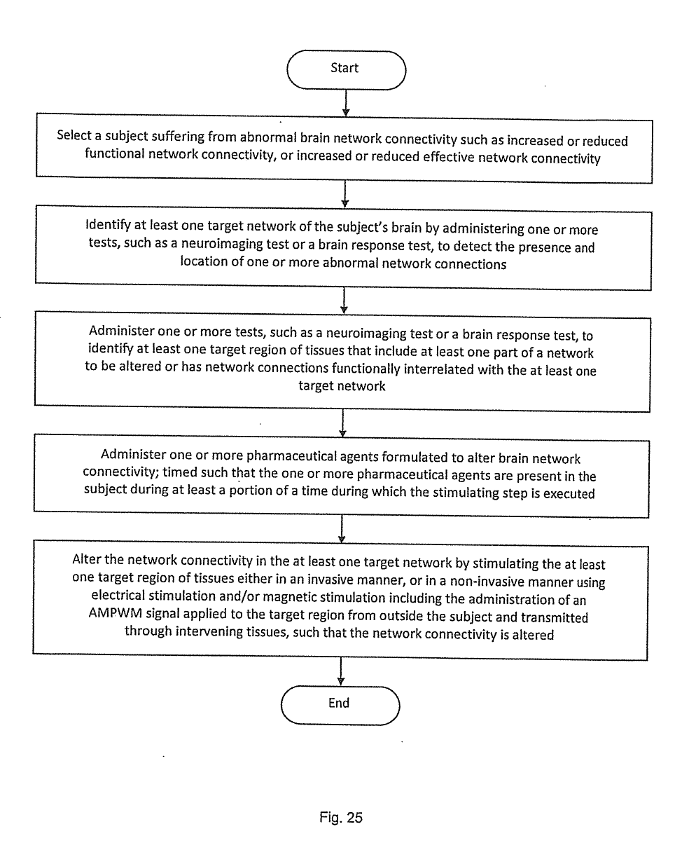

Referring to FIGS. 24-25, network connectivity in a subject's brain may also be altered, e.g., may have its network connections increased or decreased in number or in strength, by applying brain stimulation to the subject. The altering of network connectivity may be accomplished by administering a stimulation signal to tissues such that the stimulation signal is transmitted to one or more regions of the subject's brain. These regions of the subject's brain can include regions that are at least one part of a network to be altered; and can also include regions of the brain that are not part of a network to be altered, but are otherwise functionally interrelated with those regions that do possess such network. Accordingly, one of skill in the neurological arts would recognize which regions of the brain are functionally interrelated with other regions of the brain.

Where the method is directed toward altering network connectivity in a subject, the method may include identifying at least one target network of the subject's brain, identifying at least one target region of brain tissues that either include or otherwise have network connections, such as functional network connections or effective network connections, that are functionally interrelated with the network to be altered, and stimulating the at least one target region of tissues to alter the network connections. The method may further include administering one or more pharmaceutical agents to the subject to influence the altering of one or more networks.

The stimulation of a target region of tissues to alter network connectivity in a brain can be accomplished in either an invasive or a noninvasive manner Such stimulation may include at least one administration of electrical stimulation to the target region of tissues of the subject and may include at least one administration of magnetic stimulation to a target region of tissues. Stimulation may be administered in a noninvasive manner in which stimulation is applied to a target region of tissues, such as tissues in the brain, from outside the subject and transmitted through intervening tissues.

Electrical stimulation may include administration of a stimulating signal that is configured to minimize intervening tissue impedance, such as an AMPWM signal, to provide increased conduction of the stimulating signal through such tissues. The administration of an AMPWM signal may be accomplished by placing noninvasive cutaneous electrodes in an arrangement that allows for successful delivery of the AMPWM stimulating signal to a target region of tissues of the subject. This may be done using an apparatus configured to generate and deliver an AMPWM signal to the cutaneous electrodes, such as an apparatus 200 for diagnosing and treating centralized pain and a cap 33 configured to be worn on a subject's head.

To determine the presence brain network connectivity to be altered, or the location of tissues to be stimulated, one skilled in the art of medical assessment may administer and interpret one or more tests designed to detect the presence and/or location of functional network connections and/or effective network connections. Such assessments may include any one or more known neuroimaging tests. Such assessments may also include a brain response test.

The administration of a pharmaceutical may include administering at least one pharmaceutical agent formulated to alter brain network connectivity. Further, the pharmaceutical administering step is preferably timed such that the one or more pharmaceutical agents are present in the subject during at least a portion of a time during which the stimulating step is executed.

The invention is not limited in any way to the embodiments disclosed herein. In this regard, no attempt is made to show structural details of the disclosed apparatuses or process details of the disclosed methods in more detail than is necessary for a fundamental understanding of the disclosed apparatuses and methods. The description is intended only to make apparent to those skilled in the art how the several forms of the invention may be embodied in practice.

What is claimed is:

1. A method for alleviating centralized pain, the method including the steps of:
assessing the brain of a subject suffering from pain;
diagnosing one or more brain conditions associated with centralized pain;
locating at least one area of abnormal brain measure associated with an abnormal brain condition producing the centralized pain; and
applying to tissues corresponding to the at least one area of abnormal brain measure a cortical stimulation signal configured to alter the one or more brain conditions in such a way as to alleviate the centralized pain.

2. The method of claim 1 in which:
the assessing step includes taking measures of a brain condition either by direct assessment techniques known in the art such as neuroimaging, or by indirect assessment such as analysis of other biological measures;
the diagnosing step includes making brain measures to support a diagnosis of the presence of one or more brain conditions associated with centralized pain that is a result of central sensitivity;
the locating step includes locating at least one area of abnormal brain measure associated with one or more brain conditions associated with centralized pain that is a result of central sensitivity; and
the applying step includes applying a cortical stimulation signal configured to alter the one or more brain conditions in such a way as to alleviate centralized pain that is a result of central sensitivity.

3. The method of claim 1 in which:
the diagnosing step includes determining abnormal brain measures to support a diagnosis of the presence of one or more brain conditions associated with centralized pain that is a result of abnormal network connectivity involved in pain processing;
the locating step includes locating at least one area of abnormal brain measure associated with one or more brain conditions associated with centralized pain that is a result of abnormal network connectivity involved in pain processing; and
the applying step includes applying a cortical stimulation signal configured to alter the one or more brain conditions in such a way as to alleviate centralized pain that is a result of abnormal network connectivity involved in pain processing.

4. The method of claim 1 in which the applying step includes applying a cortical stimulation signal that comprises waveforms configured to minimize tissue impedance.

5. The method of claim 4 in which the applying step includes applying a cortical stimulation signal that comprises an AMPWM signal.

6. The method claim 1 in which:
the assessing step includes taking measures to determine the presence of a brain condition associated with centralized pain;
the diagnosing step includes making a determination of a brain condition associated with centralized pain; and
the locating step includes identifying at least one target region of the subject's brain where a brain condition involved in producing centralized pain originates.

7. The method of claim 6 in which the assessing step includes making brain measures using one or more neuroimaging tests directed toward determining the presence of centralized pain.

8. A method of assessing a brain to determine the presence of centralized pain, the method including the use of a brain response test (BRT) comprising one or more neuroimaging tests performed before, during and after the application of any one or more sensory stimulations to cause one or more brain responses.

9. The method of claim 8 in which the sensory stimulations include noxious, pain inducing or non-painful means of causing one or more brain responses.

10. The method of claim 8 in which a neuroimaging test includes an electroencephalogram (EEG) test.

11. The method of claim 9 in which sensory stimulations are selected from the group of sensory stimulations consisting of a tender point test, application of mechanical pressure on any part of the body, application of other forms of mechanical stimulation to any part of the body, application of an electrical stimulus, application of a heat-producing stimulus, in vivo or in vitro introduction of a chemical agent, mechanical brushing, controlled physical movements made by the subject, or forms of mental processing such as cognitive exercises.

12. The method of claim 8 in which a series of sensory stimulations are made over a period of time in such a way as to produce multiple brain responses.

13. The method of claim 12 in which a BRT includes assessing changes in brain response over a period of time.

14. The method of claim 10 in which EEG data is obtained for a period of time before, during, and after the application of any number of one or more sensory stimulations.

15. The method of claim 14 in which the EEG data is obtained over a period of time of approximately one second to 15 minutes before application of a sensory stimulation.

16. The method of claim 8 in which a record of a BRT test includes quantifying parameters associated with the one or more sensory stimulations.

17. The method of claim 16 in which recording of quantified parameters occurs at or near the time the subject reports a painful sensation.

18. The method of claim 16 in which parameters are selected from a group of parameters consisting of location of mechanical pressure, amount of mechanical pressure, parameters of other forms of mechanical stimuli, parameters of forms of electrical stimuli, parameters of forms of heat stimuli, parameters of an introduced chemical agent, parameters of brush strokes, or parameters of a mental exercise.

19. The method of claim 14 in which the recording of EEG continues for a period of time after application of each of the one more sensory stimulation applications.

20. The method of claim 19 in which the period of time is between one second and 15 minutes.

21. The method of claim 19 in which the step of recording EEG includes producing a resulting EEG data record that includes the brain response test EEG records following each one or more applications of one or more sensory stimulations.

22. The method of claim 21 in which EEG data that have minimal non-EEG signals are extracted from each EEG record to provide a period of clean EEG data sufficient to perform an EEG analysis.

23. The method of claim 22 in which the step of extracting from each EEG record includes obtaining clean EEG data that have minimal non-EEG signals and do not include any EEG data acquired during the application of any one or more sensory stimulations.

24. The method of claim 23, in which clean EEG records are mathematically analyzed.

25. The method of claim 24, in which EEG records are mathematically analyzed by selecting one or more analyses from a group consisting of time domain and frequency domain parameters.

26. The method of claim 25, in which EEG records are mathematically analyzed by selecting one or more analyses from a group consisting of voltage analysis, current analysis, voltage and current analysis, frequency spectrum analysis using Fast Fourier Transform (FFT) analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, phase analysis, various forms of network analysis and source localization of electrical activity in the brain using inverse EEG computation analysis.

27. The method of claim 24 in which mathematical analysis is used to determine one or more brain measures to support a diagnosis of the presence of one or more brain conditions associated with centralized pain.

28. The method of claim 8, in which a finding of centralized pain is made by analyzing findings from one or more BRT tests.

29. The method of claim 28, in which analyzing findings from one or more BRT tests includes performing one or more analyses to discover one or more brain conditions associated with central sensitivity or abnormal brain network connectivity associated with pain processing.

30. The method of claim 28, in which BRT EEG records are statistically compared to EEG records taken from either healthy normal individuals or individuals that are suffering from pain that is not centralized pain.

31. The method of claim 24 in which EEG abnormalities consistent with subjects suffering from centralized pain are selected from the group of abnormalities consisting of abnormal levels of EEG power, abnormal levels of coherence between at least two EEG sites, abnormal levels of phase shift between at least two EEG sites, or abnormal levels of EEG relative power in particular regions of the brain.

32. The method of claim 8, in which determination of centralized pain includes physical assessment augmented by assessment of a brain following a BRT.

33. The method of claim 28, in which assessment of a BRT includes a statistical comparison between any one or more of the subject's BRT measures and a database of like BRT measures of either healthy normal individuals, individuals suffering from pain that is not centralized pain, or individuals suffering from centralized pain.

34. The method of claim 28, in which centralized pain is diagnosed by statistically determining one or more deviations between a subject's one or more BRT measures and like BRT measures obtained from at least one healthy normal individual or at least one individual suffering from pain that is not centralized pain; then comparing the one or more deviations to like deviations detected in a sample population of subjects known to be suffering from centralized pain.

35. The method of claim 34, in which a finding of centralized pain is made when findings of a comparison of parameters in a BRT test are statistically different from like parameters in a database of healthy normal individuals, individuals suffering from pain that is not centralized pain; or statistically equivalent to like parameters in a database of individuals known to be suffering from centralized pain.

36. A method of predicting symptom severity in individuals having centralized pain, the method including the steps of:
executing BRT testing on a subject;
obtaining brain measures associated with centralized pain in the subject by analyzing findings from the BRT testing; and
correlating the brain measures to measures of symptom severity.

37. A method of determining the effect of therapeutic intervention in alleviating symptoms of centralized pain, the method including the steps of:
executing BRT testing on a subject;
obtaining brain measures associated with centralized pain in the subject by analyzing findings from the BRT testing; and
correlating the brain measures to measures of the effect of therapeutic intervention.

38. A method for determining points for application of cortical stimulation for alleviating centralized pain by analyzing a BRT.

39. The method of claim 1, in which the diagnosing one or more brain conditions associated with centralized pain step includes determining that at least one abnormal measure of the subject's brain associated with centralized pain corresponds to at least one statistically significant difference finding of a BRT test method.

40. The method of claim 39, further comprising the steps of:
repeating one or more analyses in accordance with the BRT test method following a period of therapeutic intervention on said subject;
making a statistical comparison of parameters of the repeated analyses to like parameters of the analyses of the subject's BRT measures done before the period of therapeutic intervention was started;
using these comparisons to assess the effectiveness of the therapeutic intervention, or to determine if an alternate intervention is indicated in the absence of treatment effect from a current therapeutic intervention;
using these comparisons to determine if further therapeutic intervention is indicated in the absence of any abnormal findings; and
using these comparisons to modify cortical stimulation signal parameters.

41. The method of claim 40, in which repeating one or more analyses in accordance with the BRT test method includes application of one or more sensory stimulation forms.

42. The method of claim 41, in which the one or more sensory stimulations are of types and levels used or performed before therapeutic intervention.

43. The method of claim 28, in which BRT test method data is acquired at a first location and the acquired BRT test method data is transferred via electronic means to a second location for analysis and statistical comparison.

44. The method of claim 43, in which BRT test method analysis and statistical comparison findings are transferred via electronic means from a second location to a first location.

45. The method of claim 44, in which electronic means of data transfer includes data transfer across a local area network and/or the internet.

46. The method of claim 44, in which BRT test method data is transferred via electronic means to be included in or to increase the size of databases of individuals known to be suffering from centralized pain, individuals known to be suffering from pain that is not centralized pain, and healthy normal individuals.

47. The method of claim 44, in which cortical stimulation signal parameters are determined at a second location and subsequently transferred as data via electronic means to an apparatus at a first location.

48. The method of claim 40, in which the steps of application of a cortical stimulation signal and repeat measurements and analyses of a subject's BRT test method data are continued until one or more abnormal brain conditions are modulated or alleviated, and/or alleviation of centralized pain is achieved.

49. A method for optimizing an intervention for alleviating centralized pain, the method including the steps of:
collecting brain measures associated with one or more abnormal brain conditions associated with centralized pain during a time period at or near the time of a therapeutic intervention;
analyzing the brain measures by computational algorithms to determine measures and statistics associated with the brain measures; and
using the measures and statistics to modify parameters of an intervention for the purposes of optimizing therapeutic benefit.

50. The method of claim 49, in which:
the collecting step includes collecting brain measures associated with one or more abnormal brain conditions associated with centralized pain during an intervention that includes application of a cortical stimulation signal; and
the using step includes using the measures and statistics to modify parameters of the cortical stimulation signal.

51. The method of claim 49, in which:
the collecting step includes collecting data from a BRT test comprising an EEG test;
the step of analyzing data includes determining measures and statistics of EEG data; and
the using step includes using measures and statistics of the EEG data.

52. The method of claim 49, in which:
the collecting step includes collecting data from a BRT test comprising an EEG test; and
the step of analyzing data includes analyzing EEG data from two or more scalp locations to determine measures and statistics of brain network connectivity associated with centralized pain.

53. The method of claim 49, in which a comparison between one or more parameters of a cortical stimulation signal and one or more measures and statistics is made in such a way as to determine a corresponding modification to the cortical stimulation signal's parameters for the purpose of optimizing therapeutic benefit.

54. The method of claim 1 in which the cortical stimulation signal is applied using an apparatus comprising a microcontroller configured to generate signal waveforms and coupled to a signal generator circuit configured to transform the signal waveforms into desired stimulation signals and comprising any one or more circuit elements selected from the group of circuit elements consisting of a biopotential amplifier configured to measure biopotential signals, a filter circuit configured to reduce electrical noise in biopotential signals, an isolation amplifier configured to protect human subjects, an analog-to-digital interface configured to convert analog biopotential signals to digital signals, an isolated power supply configured to provide circuit power and human subject protection, a switching transistor configured to generate an amplified stimulation signal by switching on and off electrical power from the isolated power supply in response to stimulation signals received at a base of the switching transistor from the microcontroller, and an inductor configured to induce a cortical stimulation signal into a conductor.

55. An apparatus for providing a stimulation signal, the apparatus comprising a cap configured to be worn on a subject's head, and further comprising a series of interconnectable flexible bands configured to include arms and tabs that connect when placed on a head to form a means to fit the shape of the head.

56. The apparatus of claim 55, in which the flexible bands comprise substrate materials further comprising circuits and conductive pathways integral to the substrate materials.

57. The apparatus of claim 55, in which the cap is adapted to carry one or more electrodes configured to both measure one or more EEG signals and to deliver one or more cortical stimulation signals to a subject.

58. The apparatus of claim 57, in which the one or more electrodes are configured to both measure one or more EEG signals and to deliver one or more cortical stimulation signals to a subject in such a way as to assess or alleviate one or more abnormal brain conditions associated with centralized pain.

59. The apparatus of claim 57, in which the flexible bands comprise electrically conductive pathways leading to the one or more electrodes integrated into the flexible bands.

60. The apparatus of claim 57, in which the electrodes are configured for either EEG measurement or conduction of a cortical stimulation signal.

61. The apparatus of claim 60, in which the electrodes are pre-prepared and pre-gelled.

62. The apparatus of claim 55, in which the flexible bands further comprise areas of reusable self-adhesive material configured to facilitate interconnection of the flexible bands when placed on a head to form a cap.

63. The apparatus of claim 55, in which the flexible bands further comprise one or more markers that are imprinted on arms or tabs and that are located so as to aid in proper arm and tab connection and sizing to form a cap on different sizes and shapes of a head.

64. The apparatus of claim 55, in which the flexible bands further comprise one or more markers that are imprinted on arms or tabs and that are located so as to aid in proper placement of a cap on a head.

65. The apparatus of claim 59, in which the electrically conductive pathways in the flexible bands interface to electrical conductors that electrically couple to connectors at a stimulation signal interface and/or an EEG interface.

66. The apparatus of claim 55, in which the flexible bands further comprise one or more electrical circuits configured to facilitate the measurement of one or more EEG signals or the delivery of one or more cortical stimulation signals.

67. An apparatus for diagnosing and treating centralized pain, the apparatus comprising a cap configured to be worn on a subject's head, and further comprising interconnected flexible bands that form the cap and that are configured to position one or more electrodes to measure one or more EEG signals from a subject and/or deliver one or more cortical stimulation signals to a subject.

68. The apparatus of claim 67, in which the bands are configured to position the one or more electrodes consistent with a standard international 10-20 electrode positioning system for EEG measurement.

69. The apparatus of claim 67, in which the bands are configured to position the one or more electrodes consistent with a modified international 10-20 electrode positioning system having fewer electrodes than the standard international 10-20 electrode positioning system for localized or strategically limited EEG recordings.

70. The apparatus of claim 67, in which the flexible bands are configured to position the one or more electrodes in respective locations that are not consistent with the standard international 10-20 electrode positioning system.

71. The apparatus of claim 57, in which individual electrodes positioned in flexible bands are configured to both provide for EEG measurement from a subject and conduct a cortical stimulation signal to a subject.

72. The apparatus of claim 57, in which the flexible bands comprise arms and tabs configured to position one or more electrodes in such a way as to rest on a subject's skin at non-EEG measurement sites and act as ground electrodes.

73. The apparatus of claim 72, in which the flexible bands comprise arms and tabs configured to position one or more electrodes in such a way as to rest on non-EEG measurement sites.

74. The apparatus of claim 57, in which the flexible bands comprise arms or tabs that further comprise one or more electrical circuits and one or more sensors configured to provide non-EEG measures.

75. The apparatus of claim 74, in which the one or more non-EEG measures are selected from the group of measures consisting of an electrooculogram, electromyographic activity, or measures of physical motion of a subject.

76. A method for delivering one or more cortical stimulation signals to a subject, the method including the use of an apparatus comprising one or more electrodes including one or more ground electrodes positioned in flexible bands configured to include arms and tabs that connect when placed on a head to form a cap and means to fit the shape of the head.

77. The method of claim 76, in which the apparatus further comprises positioning of electrodes such that, when the cap is worn on a subject's head in a predetermined orientation, a vector path extending between the stimulating electrode and the ground electrode passes through or proximate a desired area of brain tissues to be stimulated.

78. The apparatus of claim 55, in which the cap comprises flexible material configured to cover at least a portion of a head, at least one flexible band comprising one or more materials that do not conduct electricity, and at least one flexible band comprising at least one material that does conduct electricity.

79. An abnormal brain function diagnostic and treatment apparatus comprising a cap configured to be worn on a subject's head and comprising one or more electrodes positioned to contact a subject's head when the subject is wearing the cap, and the cap further comprising a memory configured to store information.

80. The apparatus of claim 79, in which the cap comprises a transmitter connected to the memory and configured to transmit or otherwise provide the stored information to a receiver.

81. The apparatus of claim 79, in which the cap is configured to store/transmit information comprising one or more forms of information selected from the group of information forms consisting of a unique identification code for the cap, the identification of the subject the cap is intended to be used on, dates and times of use, parameters of a cortical stimulation signal to be used with the cap, the total number of times the cap has been used, or monitoring data associated with quality of use.

82. The apparatus of claim 79, in which the cap is configured to store/transmit information that includes an indication as to whether or not the cap has been used.

83. The apparatus of claim 82, in which the cap is configured to store/transmit information usable to prevent the cap from being used again.

84. The apparatus of claim 80, in which means for storing, transmitting or otherwise providing information includes one or more bar coding methods for encoding and decoding data, and where the bar code is integrated into an element of the cap.

85. The apparatus of claim 79, in which the abnormal brain function treatment apparatus further comprises a bar code reader configured to access information stored in a bar code on a cap.

86. The apparatus of claim 55, further comprising a bar code reader configured to access information stored in a bar code on a cap.

87. A method of preventing a cap from being used more than a predetermined number of times, the method including:
providing a cap identifier on the cap;
providing on the cap an allowed use indicator representing a predetermined number of allowed uses for the cap;
gaining access to stored information in the cap identifier and the cap allowed use indicator;
comparing the stored information against a database of information on caps that have previously been used;
comparing the number of allowed uses indicated in the cap allowed use indicator against the previous cap use number stored in the cap information database; and
configuring an apparatus for diagnosing and treating centralized pain to allow use of the cap only if the cap information database indicates that the previous cap use number is less than the number of allowed uses indicated by the cap allowed use indicator.

88. The method of claim 87 in which the cap identifier and cap allowed use indicator comprise information storage devices selected from the group of storage devices consisting of a bar code or an RFID chip.

89. The method of claim 87 in which information in the cap identifier and cap allowed use indicator is accessed using a reader selected from the group of readers consisting of a bar code reader or an RFID chip reader.

90. The method of claim 87, in which information about a cap's use is added to the cap information database upon use of the cap.

91. The method of claim 90, in which the cap information database is stored locally on an apparatus for diagnosing and treating centralized pain.

92. The method of claim 90, in which the cap information database is stored in a central location and accessed via a network connection.

93. The method of claim 1, in which cortical stimulation is used to alleviate centralized pain in combination with treatment of at least one other coexisting physical condition.

94. The method of claim 1, in which cortical stimulation is used to alleviate centralized pain in combination with at least one other form of treatment utilized to affect symptoms of centralized pain.

95. The method of claim 94, in which at least one other form of treatment utilized to affect symptoms of centralized pain includes administering one or more pharmaceutical agents to the subject.

96. A method for altering brain network connectivity in a subject, the method including the steps of:
identifying at least one target network of the subject's brain;
identifying at least one target region of tissues that has network connections functionally interrelated with the at least one target network; and
altering network connectivity in the at least one target network by stimulating the at least one target region of tissues.

97. The method of claim 96 wherein the step of identifying at least one target region of tissues includes identifying a target region of tissues including brain tissues that comprise at least one part of a network to be altered.

98. The method of claim 96, wherein the identifying steps include the administration of one or more tests designed to detect the presence and location of one or more network connections.

99. The method of claim 96 wherein the step of altering network connectivity includes altering functional network connectivity.

100. The method of claim 96 wherein the step of altering network connectivity includes altering effective network connectivity.

101. The method of claim 96, wherein the stimulating step includes at least one administration of electrical stimulation to the at least one target region of tissues.

102. The method of claim 96, wherein the stimulating step includes at least one administration of magnetic stimulation to the at least one target region of tissues.

103. The method of claim 96, wherein the stimulating step is performed in a noninvasive manner.

104. The method of claim 103, wherein the noninvasive manner includes stimulation applied to a target region of tissues from outside the subject and transmitted through intervening tissues.

105. The method of claim 96, wherein the stimulating step is performed in an invasive manner.

106. The method of claim 101, wherein the step of administering electrical stimulation includes administration of an AMPWM signal.

107. The method of claim 96, wherein the step of altering network connectivity includes stimulating the at least one target region of tissues such that network connectivity is increased.

108. The method of claim 96, wherein the step of altering network connectivity includes stimulating the at least one target region of tissues such that network connectivity is decreased.

109. The method of claim 101, in which the stimulation is applied using an apparatus comprising a microcontroller configured to generate signal waveforms and coupled to a signal generator circuit configured to transform the signal waveforms into desired stimulation signals and comprising any one or more circuit elements selected from the group of circuit elements consisting of a biopotential amplifier configured to measure biopotential signals, a filter circuit configured to reduce electrical noise in biopotential signals, an isolation amplifier configured to protect human subjects, an analog-to-digital interface configured to convert analog biopotential signals to digital signals, an isolated power supply configured to provide circuit power and human subject protection, a switching transistor configured to generate an amplified stimulation signal by switching on and off electrical power from the isolated power supply in response to stimulation signals received at a base of the switching transistor from the microcontroller, and an inductor configured to induce a stimulation signal into a conductor.

110. The method of claim 96, wherein altering brain network connectivity includes administering one or more pharmaceutical agents to the subject.

111. The method of claim 98, wherein the administration of one or more tests steps includes at least one neuroimaging test.

112. The method of claim 98, wherein the administration of one or more tests steps includes at least one brain response test.

113. The apparatus of claim 57, in which the one or more electrodes are configured to measure one or more EEG signals and to deliver one or more cortical stimulation signals to a subject in such a way as to alter network connectivity.

114. An apparatus for altering network connectivity, the apparatus comprising a cap configured to be worn on a subject's head, and further comprising interconnected flexible bands that are configured to position one or more electrodes to measure one or more EEG signals from a subject and/or deliver one or more cortical stimulation signals to a subject.

115. The apparatus of claim 114, in which the bands are configured to position the one or more electrodes consistent with a standard international 10-20 electrode positioning system for EEG measurement.

116. The apparatus of claim 114, in which the bands are configured to position the one or more electrodes consistent with a modified international 10-20 electrode positioning system having fewer electrodes than the standard international 10-20 electrode positioning system for localized or strategically limited EEG recordings.

117. The apparatus of claim 114, in which the flexible bands are configured to position the one or more electrodes in respective locations that are not consistent with the standard international 10-20 electrode positioning system.
